(19) United States
(12) Patent Application Publication
(10) Pub. No.: US 2006/0037091 A1
(43) Pub. Date: Feb. 16, 2006

(54) Method for producing hypertrophic scarring animal model for identification of agents for prevention and treatment of human hypertrophic scarring

(76) Inventors: Geoffrey C. Gurtner, New York, NY (US); Kirit A. Bhatt, New York, NY (US)

Correspondence Address: Michael L. Goldman;Nixon Peabody LLP, Clinton Square, P.O. Box 31051, Rochester, NY, 14603-1051, US

(21) Appl. No.: 11/135,992

(22) Filed: May 24, 2005

Related U.S. Application Data
(60) Provisional application No. 60/573,998, filed on May 24, 2004.

(51) Int. Cl. A01K 67/027; A61K 38/54

(52) U.S. Cl. 800/18; 514/12

(57) ABSTRACT

The present invention relates to a method of producing a non-human animal model of hypertrophic scarring. This involves producing an incision in a non-human animal and applying mechanical strain over the incision under conditions effective to produce hypertrophic scarring, thereby producing a non-human animal model of hypertrophic scarring. The present invention also relates to a method of determining the efficacy of an agent for prevention or treatment of a disease condition. This method involves providing a non-human animal having an incision over which mechanical strain is applied under conditions effective to produce hypertrophic scarring, administering an agent to the incision, and determining whether the agent is efficacious for prevention or treatment of a disease condition. Also provided is a non-human animal model of hypertrophic scarring. This involves a non-human animal having an incision over which mechanical strain has been applied under conditions effective to produce hypertrophic scarring.

This application claims benefit of U.S. Provisional Patent Application Ser. No. 60/573,998, filed May 24, 2004, which is hereby incorporated by reference in its entirety.

### FIELD OF THE INVENTION

The present invention relates to a method for producing a non-human animal model of hypertrophic scarring and the use of such a model for the development of agents for the prevention or treatment of hypertrophic scarring in mammals, including humans.

### BACKGROUND OF THE INVENTION

The optimal result of human wound healing would be functional and scar-free healing (Martin, P., “Wound Healing—Aiming for Perfect Skin Regeneration,” Science 276:75-81 (1997)), but this is rarely the case. Each year more than 12 million traumatic and 1.25 million burn injuries result in disfiguring and dysfunctional hypertrophic scars (Singer et al., “Cutaneous Wound Healing,” N Engl J Med 341:738-746 (1999); Singer et al., “Evaluation and Management of Traumatic Lacerations,” N Engl J Med 337:1142-1148 (1997)). Human hypertrophic scars occur following any break in cutaneous integrity, and can result in the limitation of extremity function, erosion of skeletal structure, and lifelong disability (Wilson et al., “Latissimus Dorsi Myocutaneous Flap Reconstruction of Neck and Axillary Burn Contractures,” Plast Reconstr Surg 105:27-33 (2000); Sheridan, R., “Airway Management and Respiratory Care of the Burn Patient,” Int Anesthesiol Clin 38:129-145 (2000)). Understanding the pathophysiology of hypertrophic scars is essential to developing new therapeutics for this disease and other fibrotic disorders which cause significant human morbidity and mortality. The lack of mechanistic understanding of the exuberant fibrotic process during hypertrophic scarring has stalled progress over the past 30 years and resulted in recurrence rates exceeding 75% using existing modalities of treatment (Deitch et al., “Hypertrophic Burn Scars: Analysis of Variables,” J Trauma 23:895-898 (1983)).

The etiology and pathophysiology of human hypertrophic scarring remain unknown. Several theories have been proposed to account for human hypertrophic scar formation, including mechanical strain, inflammation, bacterial colonization, and foreign body reaction (Mustoe et al., “International Clinical Recommendations on Scar Management,” Plast Reconstr Surg 110:560-571 (2000)). Unfortunately, mechanistic investigation of hypertrophic scar formation has been hindered by the absence of a reproducible animal model that demonstrates the characteristics of human hypertrophic scars (Sheridan et al., “What's New in Burns and Metabolism,” J Am Coll Surg 198:243-263 (2004)). As recently as 2004, it was stated in a major review that, “Hypertrophic scarring remains a terrible clinical problem . . . understanding the pathophysiology and developing effective treatment strategies have been hindered by the absence of an animal model.” (Sheridan et al., “What's New in Burns and Metabolism,” J Am Coll Surg 198:243-263 (2004)).

The importance of mechanical strain in hypertrophic scar formation has been suggested by a wealth of clinical observations. For centuries, surgeons have observed that the scar hypertrophy or thickening is greatest when excessive mechanical strain is placed upon a healing wound (Singer et al., “Evaluation and Management of Traumatic Lacerations,” N Engl J Med 337:1142-1148 (1997)). Most approaches to surgically revise abnormal scars act primarily to re-orient the direction of the wound edges to relieve the forces in regions with high mechanical strain, and improve hypertrophic scars (Mustoe et al., “International Clinical Recommendations on Scar Management,” Plast Reconstr Surg 110:560-571 (2000); Suzuki et al., “Proposal For a New Comprehensive Classification of V-Y Plasty and Its Analogues: the Pros and Cons of Inverted Versus Ordinary Burow's Triangle Excision,” Plast Reconstr Surg 98:1016-1022 (1996); Longacre et al., “The Effects of Z Plasty on Hypertrophic Scars,” Scand J Plast Reconstr Surg 10:113-128 (1976); Burke, M., “Scars. Can They Be Minimised?” Aust Fam Physician 27:275-278 (1998); Edlich et al., “Predicting Scar Formation: From Ritual Practice (Langer's Lines) to Scientific Discipline (Static and Dynamic Skin Tensions),” J Emerg Med 16:759-760 (1998); Suzuki et al., “Versatility of Modified Planimetric Z-Plasties in the Treatment of Scar With Contracture,” Br J Plast Surg 51:363-369 (1998); Robson et al., “Prevention and Treatment of Postburn Scars and Contracture,” World J Surg 16:87-96 (1992); Sherris et al., “Management of Scar Contractures, Hypertrophic Scars, and Keloids,” Otolaryngol Clin North Am 28:1057-1068 (1995)). Pressure therapy (e.g., Jobst stockings) has limited efficacy (Mustoe et al., “International Clinical Recommendations on Scar Management,” Plast Reconstr Surg 110:560-571 (2000); Costa et al., “Mechanical Forces Induce Scar Remodeling. Study in Non-Pressure-Treated Versus Pressure-Treated Hypertrophic Scars,” Am J Pathol 155:1671-1679 (1999); Reno et al., “In Vitro Mechanical Compression Induces Apoptosis and Regulates Cytokines Release in Hypertrophic Scars,” Wound Repair Regen 11:331-336 (2003)) and may function to reduce mechanical strain on the wound.

It is known that living cells can sense mechanical forces and convert them into biological processes, and in turn, biochemical signals are known to influence the ability of cells to sense mechanical forces (Bao et al., “Cell and Molecular Mechanics of Biological Materials,” Nat Mater 2:715-725 (2003)). At the molecular level, mechanical forces regulate numerous physiological functions, from the mechanoresponsive activities of osteoblasts and osteoclasts to pressure-related alterations of vascular smooth muscle tone (Alenghat et al., “Mechanotransduction: All Signals Point to Cytoskeleton, Matrix, and Integrins,” Sci STKE 2002:PE6 (2002)). It is conceivable that mechanical forces could also result in pathological conditions, and a wealth of clinical evidence has suggested that mechanical strain plays an integral role in the pathogenesis of numerous fibrotic conditions, including cardiac hypertrophy, glomerulosclerosis, Dupytren's contracture, pulmonary hypertension (Ingber, D., “Mechanobiology and Diseases of Mechanotransduction,” Ann Med 35:564-577 (2003)), and hypertrophic scarring (Singer et al., “Evaluation and Management of Traumatic Lacerations,” N Engl J Med 337:1142-1148 (1997)).

The cellular and molecular effects of mechanical strain on wound healing are not known. It could potentially alter the inflammatory milieu, gene expression patterns, apoptosis, proliferation, and/or recruitment of bone marrow cells. Since apoptosis has an important role in the natural progression of the phases of wound healing (Greenhalgh, D., “The Role of Apoptosis in Wound Healing,” Int J Biochem Cell Biol 30:1019-1030 (1998)), it is hypothesized that deregulation of this process contributes to the pathogenesis of hypertrophic scarring. It is possible that mechanical strain disrupts the natural progression of wound healing by directly affecting apoptosis. What is needed is a valid animal model of hypertrophic scarring that mimics human physiology so closely as to overcome the current limitations of evaluating the process of scarring in humans.

The present invention is directed to overcoming these and other deficiencies in the art.

### SUMMARY OF THE INVENTION

The present invention relates to a method of producing a non-human animal model of hypertrophic scarring. This method involves producing an incision in a non-human animal and applying mechanical strain over the incision under conditions effective to produce hypertrophic scarring, thereby producing a non-human animal model of hypertrophic scarring.

The present invention also relates to a method of determining the efficacy of an agent for prevention or treatment of a disease condition. This method involves providing a non-human animal having an incision over which mechanical strain is applied under conditions effective to produce hypertrophic scarring. The method also involves administering an agent to the incision and determining whether the agent is efficacious for prevention or treatment of a disease condition.

The present invention also relates to a non-human animal model of hypertrophic scarring. This involves a non-human animal having an incision over which mechanical strain has been applied under conditions effective to produce hypertrophic scarring.

Hypertrophic scarring commonly occurs following cutaneous wounding and results in significant functional and aesthetic defects. The pathophysiology of this process has long been unclear. The device of the present invention provides a tool for producing a valid murine model of hypertrophic scarring. The resulting scars of the model demonstrate the cardinal histopathologic features of human hypertrophic scars. Such a model has long been needed to aid in unraveling the pathophysiology of hypertrophic scarring, and for the identification of therapeutic agents for the prevention and treatment of hypertrophic scarring and other human disease conditions characterized by a pathologic over-accumulation of cells and matrix.

### BRIEF DESCRIPTION OF THE DRAWINGS

FIGS. 1A-M show the biomechanical strain device of the present invention and some results of its application as a mouse model of hypertrophic scarring. FIG. 1A shows two exemplary biomechanical strain devices of the present invention made from expansion screws and Luhr plates. The arrows on each device indicate the direction the expansion key moves to open the device. The device on the right is shown unexpanded. The device on the left demonstrates a partially expanded device. FIG. 1B is a diagram showing the placement of the biomechanical strain devices on two 2 cm linear incisions on a mouse dorsum such that the strain is perpendicular to the incision. FIG. 1C is a diagram showing the placement of the strained and unstrained (control) incisions on a mouse from which tissue was harvested for purposes of comparative histology. FIG. 1D is a photograph of the skin in the region of the unstrained incisional wound three weeks post-wounding. The unstrained region developed very little fibrosis after 3 weeks. FIG. 1E is a photograph of the skin in the region of the strained incisional wound three weeks post-wounding. The strained region developed into hypertrophic scars which were 15-fold greater in area than the unstrained region after 3 weeks. FIGS. 1F and 1G show histologically stained sections of the skin shown in FIGS. 1D and 1E, respectively. FIG. 1H is a diagram showing how the strain vector was altered so that it was in line with the incision, creating a longitudinal force that compressed the wounds. FIG. 1I is a photograph of a histological section of the region of skin subjected to a longitudinal strain for 1 week, which resulted in increased fibrosis and hyperplasia. FIG. 1J is a photograph of a mouse with two incisions running caudo-cephalo on its dorsum. A biomechanical strain device flanks each of the incisions, sutured to the mouse's dorsal skin. One of the devices will be activated (expanded) and the other will not be activated, i.e., it will serve as a control. FIG. 1K is a top view diagram of the mechanical strain device of the present invention, shown in the non-activated state. FIG. 1L is a top view diagram of the mechanical strain device of the present invention in a partially activated state. FIG. 1M is a top view diagram, showing the device in the fully activated, i.e., full expanded state.

FIGS. 2A-E show elasticity differences among species. FIGS. 2A, 2B, and 2C are von Giemsa stained sections of murine fetus (E15), murine adult, and human skin, respectively, showing very little elastin in murine fetus E15 (time when scarless healing occurs); moderate amounts of elastin in adult mouse skin; and abundant amounts of elastin in human skin. FIGS. 2D and 2E are stress-strain and tissue resting stress curves, respectively, demonstrating that there is greater intrinsic resting/recoil force in human skin compared to adult and fetal murine skin, which, in turn, demonstrates that greater forces (stress) are required to strain human tissue.

FIG. 3A is a graph of total cell counts between the strained and unstrained regions, demonstrating 25-fold greater cellularity in the strained scars (p<0.001). FIGS. 3B-H are photographs of histological sections comparing the striking similarities between human hypertrophic scars (inset) and murine hypertrophic scarring produced by the application of mechanical strain. FIG. 3B demonstrates that, although not a histological criterion, murine hypertrophic scars appear raised histologically. FIG. 3C shows a loss of rete pegs, adnexae, and hair follicles. FIG. 3D is a 4′,6-Diamidino-2-phenylindole (“Dapi”) nuclear stained section showing that hyperplasia occurs in strained regions of both murine and human hypertrophic scarring. In FIG. 3E, polarized light-Sirius red staining for collagen demonstrates a sheet-like arrangement of fibers running parallel to the skin surface. FIG. 3F is stained for CD31, an endothelial marker, and demonstrates the perpendicular arrangement of blood vessels. FIG. 3G shows fibroblasts assume an orientation that is in parallel with collagen fibers and the direction of strain. FIG. 3H shows collagen whorls, whose function/etiology is unclear in human hypertrophic scars, can also be seen in strain-induced murine scars.

FIGS. 4A-F show differences in areas and cell density between strained and unstrained scars. FIG. 4A is a graph showing total scar areas in strained regions were 20-fold greater than in unstrained regions chronically over 6 months. FIGS. 4B and 4C are histological sections of strained and unstrained murine regions. FIG. 4B shows strained murine hypertrophic scars have dense collagen deposition, while in FIG. 4C, unstrained murine wounds are seen to heal with minimal fibrosis. FIG. 4D is a graph showing that cell densities in strained scars were 2-fold greater than in unstrained scars. Nuclear staining with Dapi demonstrates higher cellular density per mm² in the strained scars, shown in FIG. 4E, than unstrained scars, shown in FIG. 4F.

FIGS. 5A-F are results of proliferation studies in strained mouse tissue. FIGS. 5A-B show BRDU staining, which demonstrates proliferating cells in the epidermis, hair follicles, and relatively fewer in the scar bed. FIG. 5C is a graph of BRDU cell counts per high power field. No significant difference was seen between the percent of proliferating cells in strained and unstrained regions. FIG. 5D is a Western Blot showing that Akt expression is greater in strained scar and skin on day 14, and decreases in unstrained skin (β-actin expression as control). FIG. 5E is a graph showing cleaved-caspase 3 antibody expression in tissue sections. Cleaved caspase 3 is significantly greater in unstrained scar at 2 weeks. FIG. 5F is a Western Blot showing that the cleaved-caspase 3 western signal is less in strained scar and skin on day 14, and greater in unstrained skin (B-actin expression as control).

FIGS. 6A-F are the results of fibroblast activity examined in a unique load device. FIG. 6A shows (top panel) there are fewer fibroblasts in the unstrained scar, but a greater percentage of cells express caspase-3 antibody signal (white arrows), than in the strained fibroblasts (bottom panel). FIG. 6B is a graph showing a 5-fold greater number of caspase 3 positive cells in the unstrained scar than strained scar. FIG. 6C is photograph of the fibroblast plated collagen lattice (“FPCL”) device, strained with increasing mechanical weights. FIG. 6D is quantitative RT-PCR of Filamin A, demonstrating increased RNA expression with increasing load. FIG. 6E shows Akt protein expression increasing over 2-fold from control to 250 mg. FIG. 6F shows the FACS results of strained fibroblasts testing for annexin. Annexin V counts decreased with increasing strain.

FIGS. 7A-N show the effects of mechanical strain on pro-apoptotic and anti-apoptotic mice histologically. The images represent a 2-week time point. FIGS. 7A-B show that strained scars are 20 fold greater than unstrained in p53−/− mice. FIGS. 7C-D demonstrate by gross histology that the strained scar tissue in p53−/− mice appear markedly elevated, while unstrained scars remain flat. There is also greater regeneration of hair in the strained areas of p53−/− mice. FIGS. 7E-F show that strained scars are 20 fold greater than unstrained in C57/B6 mice. FIG. 7G-H demonstrate by gross histology that the strained scars are hypertrophic, but not as raised as in p53−/− mice. FIGS. 7I-J show that strained scars are 6 fold greater than unstrained in Bcl2−/− mice. FIG. 7K-L demonstrate by gross histology that the strained scars in Bcl2−/− mice appear relatively flat compared to the other mouse strains. Furthermore, there is less regeneration of hair in Bcl2−/− mice, even in the strained regions. FIGS. 7M-N are graphs showing the areas between unstrained and strained scars at 2 weeks. p53 hypertrophic scars are over 2-fold and 6-fold greater than control and Bcl2−/− strained scars.

FIG. 8 is a diagram of the pathway of mechanical strain induced regulation of Akt and hypertrophic scarring. Mechanical strain or deformation by the collagen matrix results in integrin-mediated filamin A activation and actin polymerization. Actin polymerization activates focal adhesion kinase (“FAK”) and phosphatidylinositol 3 (“PI3”)-kinase/Akt pathway. Akt then regulates cell survival by inhibiting p53 mediated apoptosis (via MDM2), and inducing Bcl2 mediated survival (via Creb).

### DETAILED DESCRIPTION OF THE INVENTION

The present invention relates to a method of producing a non-human animal model of hypertrophic scarring. This method involves producing an incision in a non-human animal and applying mechanical strain over the incision under conditions effective to produce hypertrophic scarring, thereby producing a non-human animal model of hypertrophic scarring.

In one aspect of the present invention, mechanical strain is produced by attaching to the animal a biomechanical strain device capable of providing mechanical strain over the incision in one or more directions. The device is preferably capable of being firmly secured to the non-human animal, and once secured, can be manipulated to increase or decrease the amount of mechanical strain over the incision.

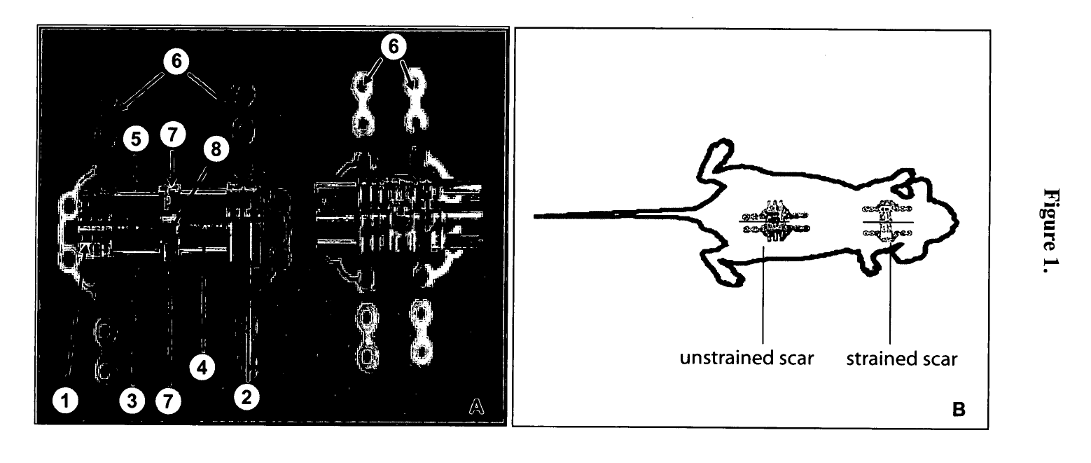

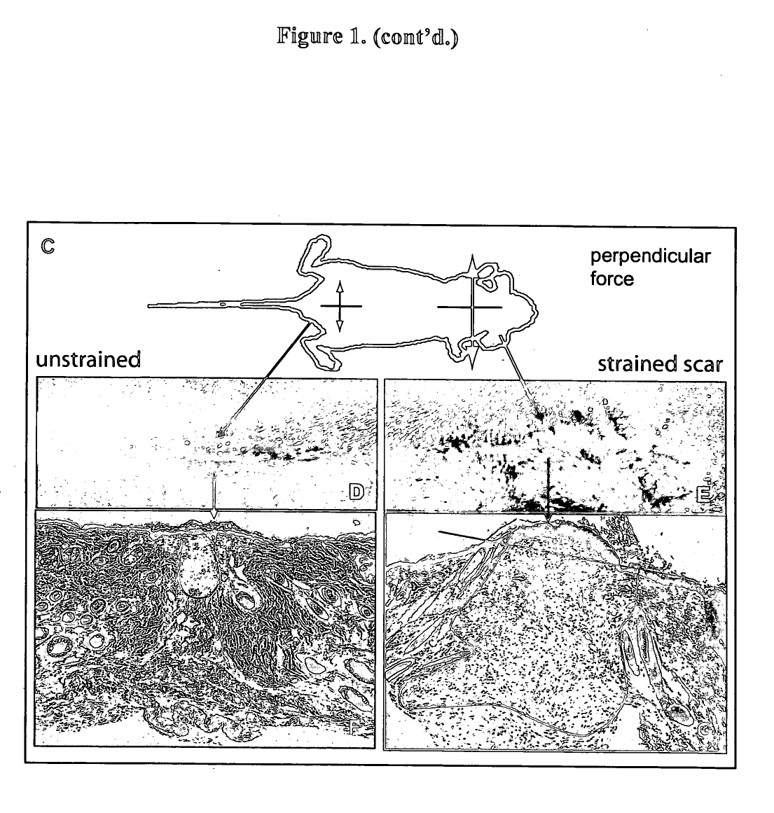

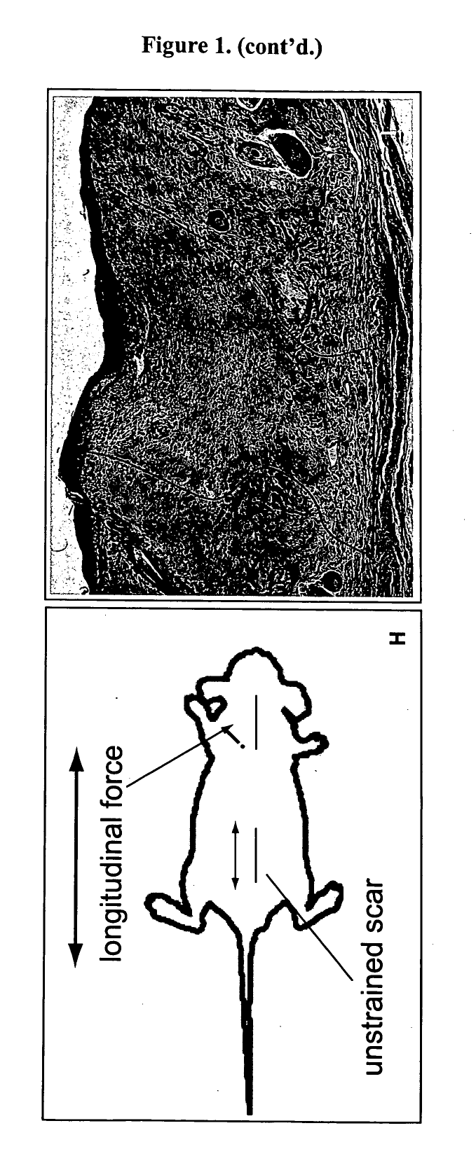

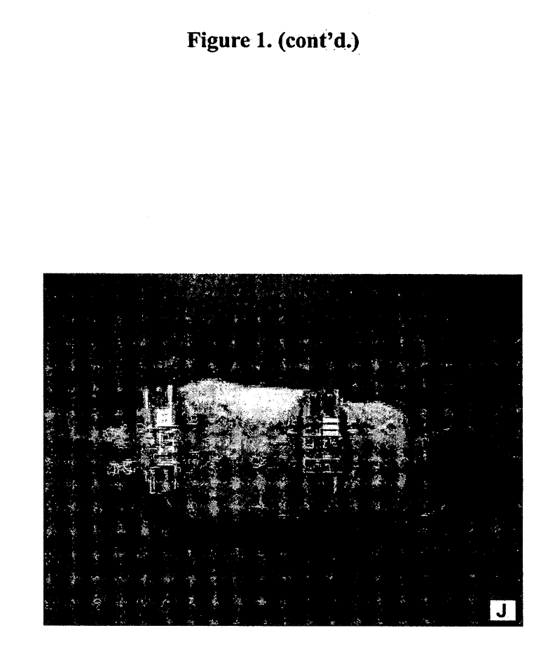

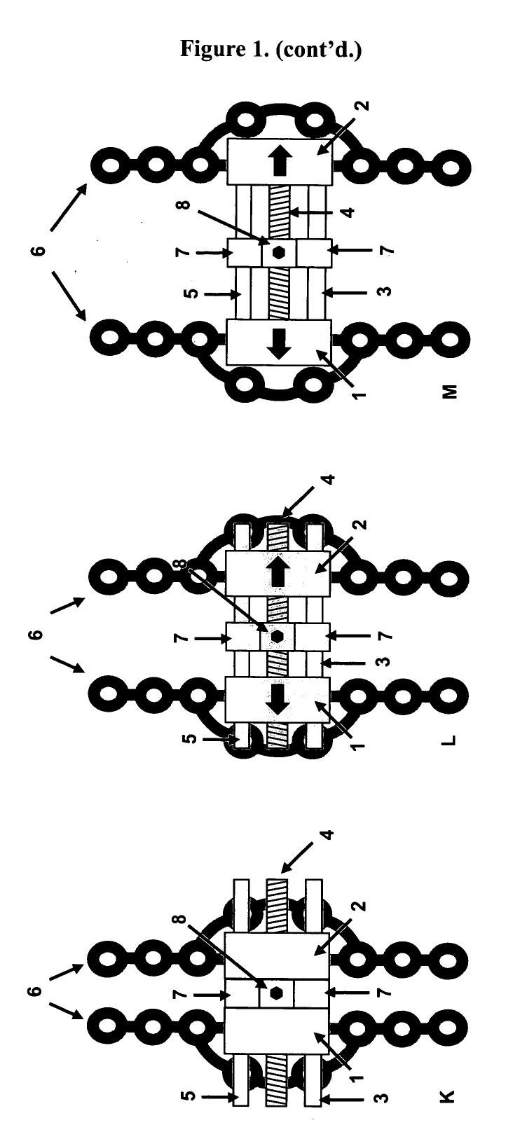

FIG. 1A shows two views of an exemplary biomechanical strain device made from expansion screws glued to Luhr plates (U.S. Pat. Nos. 5,129,903 and 5,372,589 to Luhr et al., which are hereby incorporated by reference in their entirety). The portions of the device are enumerated in the device shown on the left in FIG. 1A. The device comprises two parallel legs (6) of web-like strips consisting of hole boundaries. The legs each have a middle portion, and two end portions. The hole boundaries of the legs 6 are suitable for receiving suture or another type of biocompatible fastener for attaching the device to an animal. Also included are a first housing portion 1 and a second housing portion 2, which are parallel to one another and to the legs 6. Each housing portion includes an external surface having a top surface, a bottom surface, and a two end surfaces, and a hollow center surrounded by an internal top surface, an internal bottom surface, and two internal end surface portions. The internal top and bottom surfaces of each housing portion are threaded. The first housing 1 is secured to one of the legs, and the second 2 housing is secured to the other leg, on the top surface of the center portion of the respective leg, with the end portions of the webbing extending beyond the ends of the housing portion to allow for the hole boundaries to be used in securing the device to the animal. The external bottom portion of the housing portions are thick enough to provide the device clearance of the scar tissue that will form around and above the level of the incision. The height of the housing portions can be increased by applying additional materials to the bottom external surface of each housing portion. For example, the devices seen in FIG. 1A have two plastic plates (approximately 1 cm in length and 0.5 cm in height each) glued to the bottom of the first 1 and second 2 housing portions to provide sufficient clearance of the scar tissue that will form.

The device also includes a first guide 3, a second guide 5, and an expandable screw 4, all of which are positioned perpendicular the legs. The first guide 3, the second guide 5, and the expandable screw 4 each have an upper and lower surface, a first and a second end, and a midsection that lies between the first and second ends. The first housing 1 and second housing 2 portions encase a segment of the first guide 3, the second guide 5, and the expandable screw 4 (i.e, the guides and the screw run through the hollow center of the housing portions), but with sufficient clearance between the upper and lower surface of the housing portions and the encased guides 3, 5 and the expandable screw 4, to allow the housing portions to travel along the upper and lower surfaces of the first guide 3, the second guide 5, and the expandable screw 4. The ends of the first guide 3, the second guide 5, and the expandable screw 4 extend out of the hollow center of the first and second housing 1,2 portions. In the midsection of the expandable screw 4 is an external shaft portion 8, into which a tool, e.g., a pin, of the appropriate size can be inserted that engages the screw. The first guide 3 and the second guide 5 also each have a stop 7, over which the housing portions glide, and which help stabilize the screw 4. The stops 7 comprise a ring through which the respective guide rod runs, and are preferably secured (e.g., by welding) to the guide rod. The stops have a slightly raised top surface, and are not connected to, but abut the external shaft 8 of the screw 4. When the pin is rotated in the shaft 8, the screw 4 is turned. The screw is threaded clockwise from the midsection to one end, and counter clockwise from the midsection to the other end, as shown in FIGS. 1K-M, thus the turning of the screw causes the first 1 and second 2 housing portions to be displaced relative to one another, i.e, to move away from the midsection of the device, in opposing directions, towards the ends of the device.

The device on the right in FIG. 1A is shown in a non-activated, or closed, state. The device on the left in FIG. 1A is shown in a partially extended, or partially open, state. In one aspect of the present invention, to produce hypertrophic scarring in a non-animal model the device is placed over an incision on an animal, with the legs of the device parallel to and straddling the incision, while the device is in an unexpanded condition. The legs of the device are firmly secured to the skin of the non-human animal using the holes in the webbing of the legs for fastening. FIG. 1B shows a non-activated device attached over the caudal incision of the mouse. A pin is rotated in the external shaft of the expandable screw, in the direction shown by the arrow on the second housing portion in FIG. 1A, engaging the screw and causing the first and second housing portions to be displaced from the midsection of the device and travel along the first and second guides towards the outside edges of the device. The greater the rotation of the pin, the greater the displacement of the first and second housing portions from one another. This movement is shown incrementally in FIGS. 1K-M. FIG. 1K shows a device in the non-activated state, with the first and second housing portions non-extended. When the screw is activated by inserting the appropriate tool into the opening in the external shaft 8 of the expandable screw 4 and rotated, the screw turns, and the first 1 and second 2 housing portions are displaced from one another, each one moving in the direction indicated by the thick arrows in FIG. 1L. In FIG. 1M, the device is shown fully activated, with the first 1 and second 2 housing portions fully displaced from one another, at the lateral edges of the device.

In FIG. 1B the activated device seen on the left in FIG. 1A and FIGS. 1L-M, is shown positioned over the cephalad incision of the mouse, which will cause hypertrophic scarring of the skin in the region of the incision due to the mechanical strain applied by the device.

The device of the present invention for producing hypertrophic scarring in a non-human animal model is not in any way limited to the construct shown in FIG. 1A. The device may be constructed of any material, including, without limitation, metal, plastic, plastic polymers, wood, paper (including cardboard), and glass. Any means may be used to cause the displacement of the first 1 and second 2 housing portions from one another is suitable. In one aspect, the device is incrementally extendable, as shown in FIGS. 1K-M. In another aspect, the device may be fashioned to move to a set distance rather than incrementally, to provide a rep-determined degree of distraction (strain). Attachment of the device to the animal can be made using any type of fastener or adhesive that is suitable for use on the skin of a live animal.

FIG. 1B is a diagram of an exemplary embodiment of this aspect, where the device of the present invention, shown in FIG. 1A, is attached to the dorsum of a mouse having a cephalad incision and a caudal incision. The device is attached by suturing it to the dorsal skin of the animal model such that the device is over the incision, as shown in FIG. 1J. When the device is activated (expanded) to increase the distance between the first and the second ends of the device, strain is applied to the skin over which the device is placed. As described in greater detail in the Examples, infra, the application of strain over the incisional wound produces hypertrophic scarring in the animal. In one aspect of the present invention, the mechanical strain is re-applied in a cyclical fashion, every other day. For example, on the day post-wounding that the application of strain begins, the device is manipulated to apply the desired degree of strain over the incision. The following day (i.e., approximately 24 hours later), the strain is relaxed, so that little or no strain is applied over the wound. After a day in the relaxed state, strain is again applied to the region of the incision. When an expandable device is used that provides a variable degree of mechanical strain, such as that shown in FIG. 1A, relaxation of the strain can occur by returning the biomechanical strain device to its unexpanded, or nearly unexpanded state. It is also possible to relax the strain by removing the device or other source of strain from the animal for the desired period of relaxation of strain.

FIG. 1J shows an exemplary non-human animal model of hypertrophic scarring of the present invention. One incision is sufficient to create the non-human animal model of hypertrophic scarring of the present invention. However, a second incision, as seen in FIG. 1J, that has a mechanical strain device placed over an incision, where the device is left in its unextended position, provides a suitable “unstrained” model, i.e., an experimental control, in the same animal.

In this and all aspects of the present invention, attachment of the mechanical strain device to the animal can be carried out by surgical sutures, dermal staples, or any other biologically compatible adhesive or fastener that firmly secures the device to the skin of the animal.

In this and any aspect of the present invention, the mechanical strain may be applied in one or more direction relative to the orientation of the incision on the animal. When the strain is applied in one direction, the direction is perpendicular to or parallel (longitudinal) to the line of the incision. This generally involves orienting the device over the incision to produce strain along the desired line, or vector. When the device is oriented perpendicular to the incision, the strain force is perpendicular to, i.e., along the sides of the incisional wound, and the edges of the wound are pulled apart, as shown in FIG. 1C. When the device is oriented parallel to the incision, the strain force is longitudinal, and the edges of the incisional wound are pushed together, as shown in FIG. 1H. Either orientation is suitable for producing hypertrophic scarring in a non-human animal model. FIG. 1J shows an embodiment in which the activated device will apply mechanical strain in a direction perpendicular to the direction of the incisions in the mouse.

In another aspect, the device is capable of applying strain in both a perpendicular and a parallel direction (relative to the incisional wound). The strain may be applied in both directions simultaneously or may be applied alternately in one direction and then the other, with or without a period of relaxation of strain between alternate applications. Strain may also be applied over multiple vectors at a single time, in any combination thereof.

In yet another aspect, two devices are employed, with a first device oriented to provide mechanical strain in direction parallel to the incision, and a second device oriented to provide mechanical strain in a direction perpendicular to the incision.

The presence and production of hypertrophic scarring is determined visually, histologically, and morphometrically, using methods well known in the art, including, but not limited to, those described herein infra (in the Examples).

Suitable animals for this aspect of the present invention are any non-human mammals, including, without limitation, mice, rats, hamsters, gerbils, rabbits, cats, and dogs.

The present invention also relates to a non-human animal model of hypertrophic scarring that has an incision over which mechanical strain has been applied under conditions effective to produce hypertrophic scarring. The non-animal model of the present invention is prepared using the method described herein to produce mechanical strain and hypertrophic scars. In a preferred embodiment, the non-animal hypertrophic scarring model of the present invention develops hypertrophic scarring having the characteristics of human, or human-like hypertrophic scarring. These characteristics are well-known in the art, and include, without limitation, those described herein infra (see Example 11).

The present invention also relates to a method of determining the efficacy of an agent for prevention or treatment of a disease condition. This method involves providing a non-human animal having an incision over which mechanical strain is applied under conditions effective to produce hypertrophic scarring. The method also involves administering an agent to the incision and determining whether the agent is efficacious for prevention or treatment of a disease condition. An agent is considered efficacious when there is a decrease in the presence of hypertrophic scarring in the non-human animal model receiving the agent compared to an animal model that has not received the agent. “Agent” as used herein is also meant to encompass one or more agents, and any combination thereof.

In this aspect of the present invention, a suitable non-human animal model is one that has been made according to the method described herein.

The administration of a suitable agent to the incision is preferably dermal, i.e., the agent to be tested is topically applied to the wound. However, the agent can also be administered orally, parenterally, subcutaneously, intravenously, intramuscularly, intraperitoneally, by intranasal instillation, or by application to mucous membranes, such as that of the nose, throat, and bronchial tubes.

Suitable agents for administration in this aspect of the present invention are those that can interfere with the process of hypertrophic scar formation. As described in greater detail in the Examples, infra, apoptosis is an important factor in hypertrophic scar formation. When the effects of mechanical strain on scarring in animals with altered apoptotic pathways was examined, it was concluded that mechanical strain on healing murine wounds produces human-like hypertrophic scars by inhibiting cellular apoptosis through upregulation of the pro-survival marker, Akt, during the proliferative phases of wound healing. Therefore, suitable agents for use in the prevention and treatment of hypertrophic scar formation include, without limitation, pro-apoptotic agents, i.e, agents that increase apoptotic activity at the site of the incision, for example, the pro-apoptotic agent BH3I-1/BH3I-2, and other agents that are capable of upregulating the expression of apoptotic molecules at the incisional site. BH3I-1 (5-(ρ-Bromobenzylidine-α-isopropyl-4-oxo-2-thioxo-3-thiozolidineacetic acid (C₁₅H₁₄BrNS₂O₃), and BH3I-2 (3-iodo-5-chloro-N-[2-chloro-5((4-chlorophenyl)sulphonyl)phenyl]-2-hydroxybenzamide ((C₁₉H₁₁Cl₃INO₄S) are known to individually be capable of inducing apoptosis, therefore, they are suitable for use individually in this aspect of the present invention. Also suitable are any molecules that are analogues of BH3I-1 and BH3I-2, for example, BH3I-1”, (5-Benzylidine-α-isopropyl-4-oxo-2-thioxo-3-thiozolidineacetic acid (C₁₅H₁₅NO₃S₂), an analog of BH3I-1 that is also known to induce apoptosis. Also suitable are compounds comprising these molecules in any combination, for example, BH3I-1/BH3I-2, or any combination of individual molecules.

Also suitable in this aspect of the present invention are agents capable of blocking the activity of anti-apoptotic molecules. This includes, for example, agents that can down-regulate Akt or other pro-survival factors, or inhibit or down-regulate transcription, translation, expression or the activity of any members of the anti-apoptotic Bcl2 family. The efficacy of any agent is determined by a reduction of hypertrophic scarring at the wound (incision) site of the animal model of the present invention to which a test agent has been administered compared to an animal model that has not received the test agent. Reduction of hypertrophic scarring is determined visually, histologically, and morphometrically, using methods well known in the art, some of which are described herein infra (in the Examples).

Because the apoptotic pathway has been implicated in the development of other fibrotic disorders, agents that show efficacy in the prevention or reduction of hypertrophic scarring as determined by administration of the agent to the non-human animal model of hypertrophic scarring of the present invention will also be good candidates for use in the prevention and treatment of other diseases. In particular, such agents may be efficacious for prevention or treatment of diseases or disease conditions characterized by cellular hypertrophy and the pathological accumulation of cells and matrix. These diseases include, without limitation, fibrotic disorders, cancer tumors, glomerulosclerosis, congestive heart failure, cardiac hypertrophy, Dupytren's contracture, pulmonary hypertension, and atherosclerosis. Thus, the non-human animal model of hypertrophic scarring provided in the present invention has applicability as a model for the determination of efficacious therapeutics for a variety of human disorders.

In one aspect of the present invention, an agent is administered to test its efficacy in preventing hypertrophic scarring. In this aspect, it is highly preferable that the test agent be administered before the incision (or injury) enters into the proliferative stage of wound healing. This aspect is described in greater detail in the Examples, infra.

The present invention also relates to a non-human animal model of hypertrophic scarring. This involves a non-human animal having an incision over which mechanical strain has been applied under conditions effective to produce hypertrophic scarring. In this aspect of the present invention, an incision is made in a non-human mammal, including, but not limited to a mouse, rat, hamster, gerbil, rabbit, cat or dog. Following the creation of the incision, the incision may be sutured or may be left unsutured. Mechanical strain is applied over the incision by use of a device that is capable of applying biomechanical strain in one or more directions relative to the direction of the incision.

In one aspect of the present invention, the non-human animal model exhibits hypertrophic scarring produced by the application of mechanical strain that includes the characteristics of human hypertrophic scarring. This is an animal that has had strain applied in a cyclical fashion, approximately every other day, to provide a period of strain followed by period of relaxation of strain, carried out as described above, and in the Examples, infra.

The following examples are provided to illustrate embodiments of the present invention, but they are by no means intended to limit its scope.

### EXAMPLES

### Example 1

### In Vivo Strain

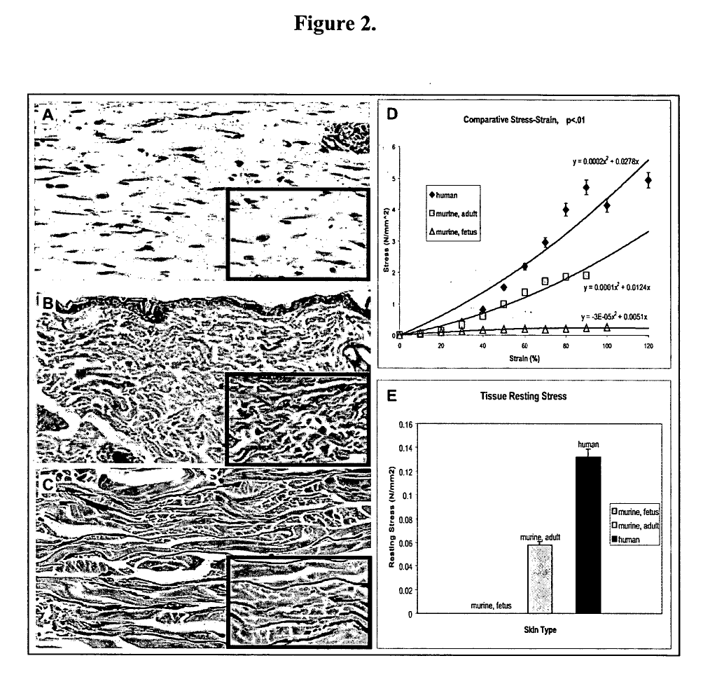

Four week old C57/BL6 mice were first acclimated and housed under standard conditions, using protocols approved by the New York University Animal Care and Use Committee. Mouse strains B6.129S2-Trp53^{tm1Tyj}/J (anti-apoptotic) and B6.129S2-Bcl2^{tm1Sjk}/J (pro-apoptotic) (Jackson Laboratory, Bar Harbor, Me.) were used for the knockout studies. Two 2 cm linear full-thickness incisions (1.25 cm apart) were made on the dorsum of the mouse and then reapproximated with 6-0 nylon sutures. On post-incision day 4, the sutures were removed from the scars, and two biomechanical strain devices, shown in FIG. 1A, were carefully secured with 6-0 nylon sutures, as shown in FIG. 2B. The biomechanical strain devices were constructed from 22-mm expansion screw (Great Lakes Orthodontic Products, Tonawanda, N.Y., USA) and Luhr (Stryker-Leibinger Co, Freiburg, Germany) plate supports, as shown in FIG. 1A. One wound served as an internal control, with the device not activated, while mechanical strain was applied over the other wound every other day by expanding the device 2 mm or 4 mm. During the periods in which strain was not applied, the natural elongation of skin over time due to an external load resulted in a steady decline in the force on the wounds. The strain was re-applied in a cyclical fashion, every other day.

The stress-strain relationship was evaluated in mouse skin using the Instron Mini 44 and a simple mathematical equation was derived to quantify the stress applied to mouse wounds. Prior to applying strain, two points were identified on either side of the scars. The two points were distracted 2 mm on post-incision day four, and 4 mm thereafter. This resulted in 11 and 18% strain, respectively. The stresses on the wounds were 1.5 and 2.7 N/mm², respectively (Stress=0.0013*(Strain²)+0.01241*(Strain)). The forces applied to the wounds from investigator to investigator were standardized based on the strain experienced by the wounds.

Tissue consisting of the scar and surrounding skin was harvested. At the designated time points, the mice were sacrificed and the harvested tissues were fixed in 10% formalin or snap frozen in liquid nitrogen for immunohistochemistry, or preserved in TriReagent (Sigma-Aldrich, St. Louis, Mo.) for RNA analysis.

### Example 2

### In Vitro Strain

In order to study the molecular mechanisms of mechanical strain on a cellular level, human (HTERT-BJ1, Clonetech, Palo Alto, Calif.) and primary murine fibroblasts was examined in vitro. A novel in vitro model as designed and described by Holmes (Costa et al., “Creating Alignment and Anisotropy in Engineered Heart Tissue: Role of Boundary Conditions in a Model Three-Dimensional Culture System,” Tissue Eng 9:567-577 (2003); Knezevic et al., “Isotonic Biaxial Loading of Fibroblast-Populated Collagen Gels: A Versatile, Low-Cost System for the Study of Mechanobiology,” Biomech Model Mechanobiol 1:59-67 (2002); Zimmerman et al., “Structural and Mechanical Factors Influencing Infarct Scar Collagen Organization,” Am J Physiol Heart Circ Physiol 278:H194-200 (2000), which are hereby incorporated by reference in their entirety) was utilized. Briefly, this model maintains fibroblasts in a three-dimensional matrix (fibroblast plated collagen lattice, “FPCL”), thereby closely resembling an in vivo environment. Ten million fibroblasts are embedded in a three-dimensional collagen lattice and exposed to a quantifiable, reproducible, and graded amount of strain. Replicate control samples were maintained under static conditions with no applied strain.

### Example 3

### Cell Culture

Human HTERT-BJ1 cells were grown in DMEM (Invitrogen, Carlsbad, Calif.) supplemented with 10% fetal bovine serum (Invitrogen, Carlsbad, Calif.) and 1% antimycotic/antibiotic at 37° C. in a CO₂ incubator. The cells were serum-starved for 18 h prior to conducting the in vitro experiments.

### Example 4

### Quantitative Real-Time RT-PCR

Total RNA was extracted from cultured cells or homogenized tissue with Tri-Reagent (Sigma, St. Louis, Mo.) and purified by an RNeasy kit (Qiagen, Valencia, Calif.). RNA PCR core kit (Applied Biosystems, Foster City, Calif.) was used to construct the template cDNA for real-time PCR (Cepheid Smartcycler) using Platinum SYBR Green Supermix-UDG (Invitrogen, Carlsbad, Calif.). Relative quantification of PCR products was calculated after normalization to β-actin or glyceraldehyde-3-phosphate dehydrogenase. Results represent three independent experiments. Products were sequenced to confirm their identity.

### Example 5

### Western Blot

After protein standardization, 50 μg of protein was run on a 12.5% polyacrylamide gel and blocked overnight using casein in TBS (Pierce Chemical Pierce, Rockford, Ill.). Protein was then transferred to a nitrocellulose membrane (Hybond-ECL; Amersham Biosciences, Piscataway, N.J.) at 100V for 45 minutes. The samples were then subjected to immunoprecipitation with anti-Akt (Cell Signaling Technology, Inc., Beverly, Mass.) followed by phosphorylation with the appropriate secondary antibodies (Cell Signaling Technology, Inc., Beverly, Mass.). Detection was completed with ECL-Plus detection reagent and Hyperfilm chemiluminescence film (Amersham Biosciences, Piscataway, N.J.).

### Example 6

### Histology

Routine hematoxylin and eosin and picrosirius red staining (Junqueira et al., “Picrosirius Staining Plus Polarization Microscopy, a Specific Method for Collagen Detection in Tissue Sections,” Histochem J 11:447-455 (1979), which is hereby incorporated by reference in its entirety) to enhance polarization of collagen fibers was performed on 5 μm thick paraffin-embedded sections. The differences in the architecture of the experimental versus the control scars were assessed using a polarizing microscope (Olympus BX51, New York, N.Y.).

### Example 7

### Immunohistochemistry

Standard light microscopy immunohistochemistry using the immunoperoxidase staining technique was performed on 4 μm thick paraffin-embedded tissue sections. Since the protocols for the various primary antibodies differed, a generalized protocol is presented here. Briefly, the sections were dewaxed and endogenous peroxidase activity was quenched with 3% hydrogen peroxide for 10 minutes, followed by blocking serum for 1 hour. The primary antibodies used included cleaved caspase-3 (1:200, Cell Signaling Technology, Inc., Beverly, Mass.), CD31 (1:100, Molecular Probes, Inc., Eugene, Oreg.), CD68 (1:100, Serotec, Raleigh, N.C.), CD45 (1:100, BD Pharmingen, San Diego, Calif.), PCNA (1:100, Abcam, Cambridge, Mass.); incubation was done on paraffin sections. The tissue sections were incubated with the primary antibody diluted in the blocking serum overnight at 4° C. After thorough washing with PBS, the sections were incubated with the secondary antibody for 30 minutes at room temperature. This was followed by incubation with the ABC (Vectastain elite ABC kit, Vector Laboratories, Burlingame, Calif.) complex for 1 hour at room temperature. Sections were thoroughly washed with PBS after each step. The sections were then incubated in 0.05% diaminobenzidine (DAB) until the brown substrate was formed, rinsed in distilled water, counterstained with hematoxylin (Vector, Burlingame, Calif.), dehydrated, and mounted in VectaMount (Vector, Burlingame, Calif.). BRDU (Zymed Laboratories, San Francisco, Calif.) staining was performed according to the Zymed manufacturer's recommendations. As negative controls for the staining procedure, sections were incubated with the blocking serum only, omitting the primary antibody; the rest of the protocol was kept unchanged. Nonspecific brown cellular staining was not observed in any of the sections used as negative controls for the immunohistochemistry. Total cellularity was counted based on total Dapi (nuclear counterstain) counts. All histological measurements were independently calculated blindly by two independent observers.

### Example 8

### Morphometry

Total scar areas were evaluated on digital images (Olympus BX51, New York, N.Y.) of hematoxylin-eosin stained sections, using SigmaScan image analysis software (Aspire Software International, Leesburg, Va.) at 100× objective, unless otherwise noted. The effects of mechanical strain over a one month period were evaluated at weekly time points. The images were evaluated blindly by two independent observers and no difference was found in the data. The results are presented as mean+/−SD.

### Example 9

### Statistical Analysis

The animal studies involved 3-6 mice for each treatment group. Data were analyzed using SigmaStat 2.0 (Aspire Software International, Leesburg, Va., USA). Statistical analysis was carried out using two-tailed Student's unpaired t test or an analysis of variance (ANOVA). All data are presented as mean+/−SEM. Probability values of P<0.05 were considered significant.

### Example 10

### Biomechanical Properties of Human and Mouse Skin Affect Scar Formation

It is well known that humans often develop exuberant dermal scarring, whereas mice normally do not. In addition, it has been established that mammalian mid-gestation fetuses heal with no scar formation at all. Qualitatively, in contrast to human skin, the skin enveloping a mouse is loose, with little recoil/elasticity, and fetal skin is almost gelatinous in texture. To quantify the qualitative biomechanical differences among the groups, dynamic tension was examined, which is a barometer of skin elasticity (Edlich et al., “Predicting Scar Formation: From Ritual Practice (Langer's Lines) to Scientific Discipline (Static and Dynamic Skin Tensions),” J Emerg Med 16:759-760 (1998), which is hereby incorporated by reference in its entirety). Young's modulus, which is the ratio of stress over strain and a property of stiffness, and resting strain are greatest in human skin, and progress from mouse to fetal skin indicating a linear decrease in elasticity, as shown in FIGS. 2D-E. Histological analysis for elastin fibers (von Giemsa stain) in human, adult mouse, and murine fetal (E15; scarless healing) skin suggested that differences in elastin content were responsible for these differences in biomechanical properties. Elastin fibers were abundant in human breast skin, moderate in adult murine skin, and rare in fetal skin, as shown in FIGS. 2A-C. The correlation between biomechanical properties and scar formation led to the examination of whether the baseline biomechanical forces of skin are in part responsible for the different scarring patterns observed (humans>murine skin>fetal skin). To test this hypothesis a technique was developed to augment the biomechanical forces on murine skin to reproduce the forces normally experienced by human skin.

### Example 11

### Effect of Mechanical Strain on Murine Wound Healing

To directly examine the effect of human levels of strain on healing murine wounds, a simple strain device was developed that could be applied to incisional wounds, shown in FIG. 1A. Two separate full-thickness wounds were created on each mouse, shown in FIGS. 1B-C, and mechanical force was applied to one in a cyclical fashion beginning on day 4, which corresponds with the initiation of the proliferative phase of wound healing. The other wound was not strained and served as an internal control. Pilot studies had demonstrated that at day 4 re-epithelialization had occurred and the risk of wound dehiscence (rupture) was minimized. Prior experiments also demonstrated that this range of forces (6-10 N/mm²) would affect the tissues at the cellular level without exceeding the breaking limits (19 N/mm²) of the wound.

The timing of strain application was critical to the formation of hypertrophic scars. Strain during the earlier inflammatory phase (days 1-3) resulted in wound breakdown; strain during the proliferative phase of wound healing (day 3-10) resulted in exuberant scars, whereas strain during the remodeling phase after day 10 had little effect on subsequent scar formation. The unstrained wound healed with minimal scarring, shown in FIG. 1D, but the strained region developed into human-like hypertrophic scars with increased volume and cellularity, as shown in FIG. 1E (Linares et al., “The Histiotypic Organization of the Hypertrophic Scar in Humans,” J Invest Dermatol 59:323-331 (1972); White, C., Textbook of Dermatopathology. New York: McGraw Hill. 349-355 pp. (2004); Ehrlich et al., “Morphological and Immunochemical Differences Between Keloid and Hypertrophic Scar,” Am J Pathol 145:105-113 (1994), which are hereby incorporated by reference in their entirety). Histologically, the unstrained scar is small, as shown in FIG. 1F, whereas the strained scar is 10-20 fold larger, shown in FIG. 1G. There were no differences in scar formation when the strain device was activated over the cephalad or caudal wound, as might be expected to occur if Hox gene differences were responsible (Chauvet et al., “Distinct Hox Protein Sequences Determine Specificity in Different Tissues,” Proc Natl Acad Sci USA 97:4064-4069 (2000); Stelnicki et al., “Bone Morphogenetic Protein-2 Induces Scar Formation and Skin Maturation in the Second Trimester Fetus,” Plast Reconstr Surg 101:12-19 (1998); Stelnicki et al., “HOX Homeobox Genes Exhibit Spatial and Temporal Changes in Expression During Human Skin Development,” J Invest Dermatol 110:110-115 (1998); Stelnicki et al., “The Human Homeobox Genes MSX-1, MSX-2, and MOX-1 are Differentially Expressed in the Dermis and Epidermis in Fetal and Adult Skin,” Differentiation 62:33-41 (1997), which are hereby incorporated by reference in their entirety). In short, by applying human levels of strain in healing murine wounds, human-like hypertrophic scarring was produced.

To eliminate the possibility that what was actually being produced was a gradual wound dehiscence or separation, the vector of the mechanical force was altered so that it was applied parallel to the incision. This orientation resulted in forces which acted to approximate the wound edges together, shown in FIG. 1H. Thus, as the longitudinal force increased, the compressive force bringing the two wound edges together also increased. After a short exposure (7 days) to longitudinal mechanical strain, increased hyperplasia and fibrosis was again observed compared to the unstrained wounds, as shown in FIG. 1I. The total scar area of “longitudinal” strain was 0.87 mm², “perpendicular” strain was 1.12 mm², and the unstrained wound was 0.18 mm², a five-fold difference. These studies demonstrate that mechanical strain alone applied for a single seven day period is sufficient to generate hypertrophic scarring in mice.

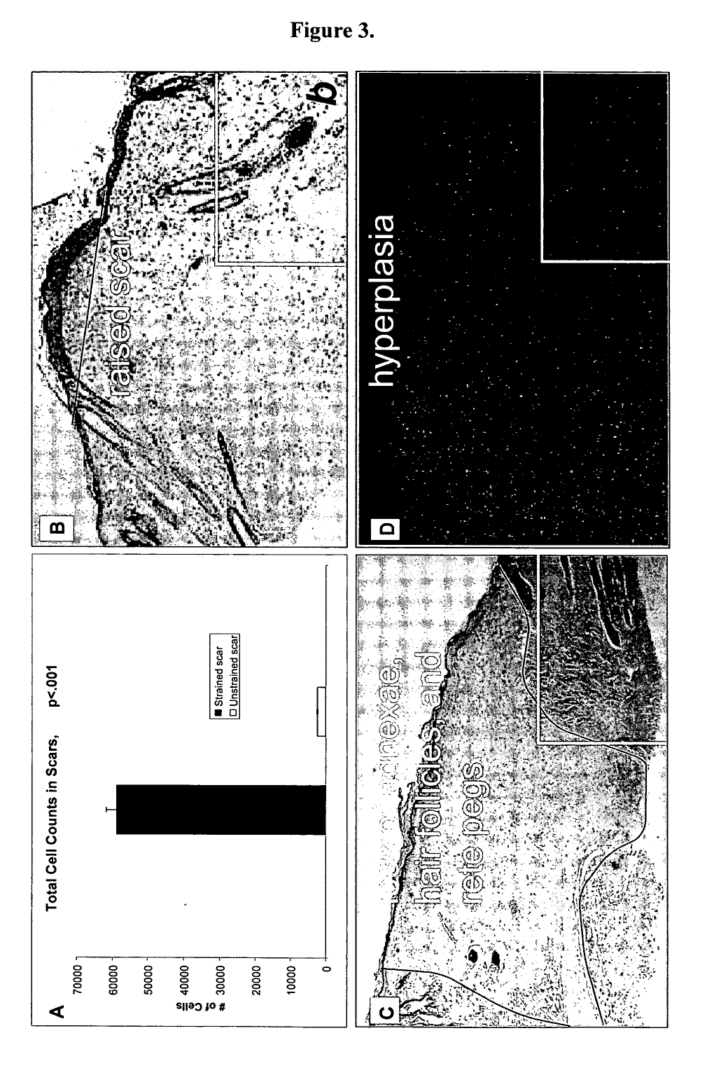

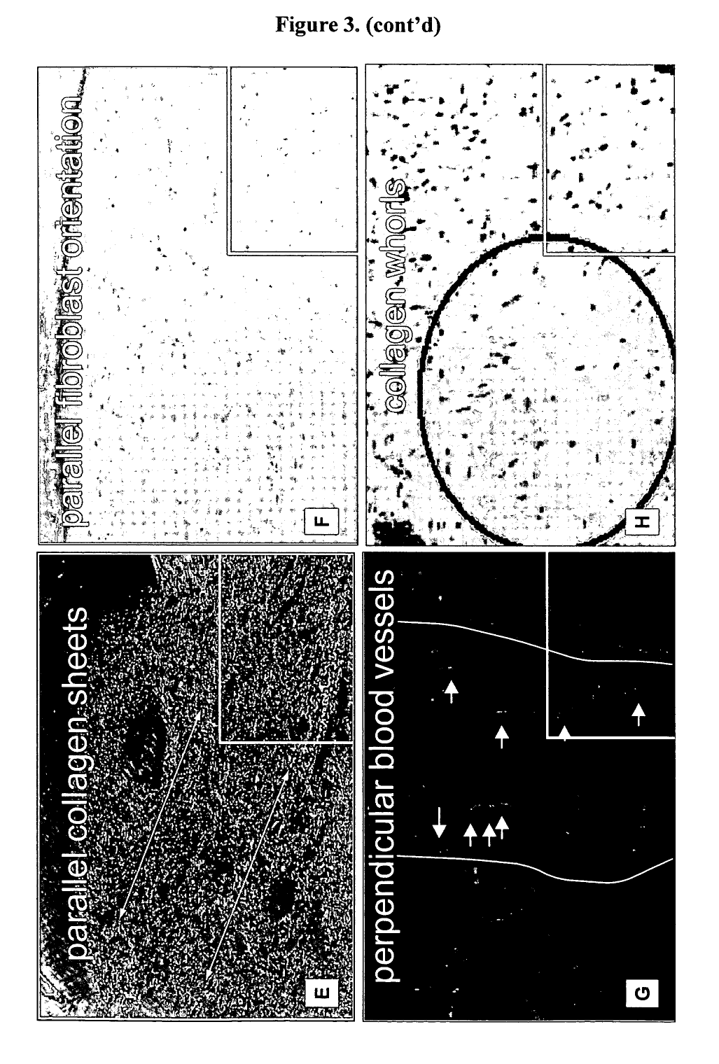

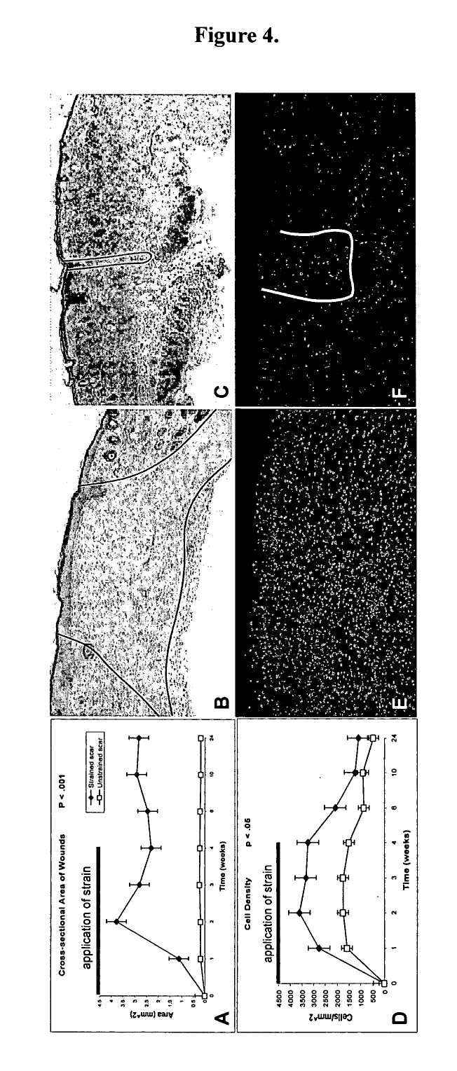

Although mechanical strain was applied early, the gross changes were not visible until after week 1. By four weeks, the total cell counts in the strained scars was 25-fold greater than in the unstrained scars (p<0.05), shown in FIG. 3A. The total scar area was increased twenty-fold in the strained region. At two weeks, shown in FIGS. 4B-C, and following this, a modest decrease in hypertrophic scar areas from week 2 to week 24 (3.7 mm² to 2.8 mm²) was observed. The unstrained scars remained stable during this time with an area of 0.25 mm². Even with this, at 6 months, there was still a 10-fold difference between the hypertrophic scar region and controls, shown in FIG. 4D. These data suggest that mechanical strain applied for a brief duration (7 days) during a vulnerable period has a chronic effect on scar morphology, persisting for up to 6 months. This suggests that targeting therapeutics to this 2 week vulnerable window could have a lasting effect on human hypertrophic scars.

### Example 12

### Mechanical Strain-induced Hypertrophic Scars in Mice Features Characteristics of Human Hypertrophic Scars

While it was evident that mechanical strain resulted in abnormal scar formation in mice, it was unclear whether histologically it resembled classic human hypertrophic scars. Abnormal scarring in humans is divided into hypertrophic scarring or keloid formation. Keloids are less common, and have a genetic component that limits them to <6% of the population, primarily the African-American and Asian populations (Deitch et al., “Hypertrophic Burn Scars: Analysis of Variables,” J Trauma 23:895-898 (1983); Marneros et al., “Genome Scans Provide Evidence for Keloid Susceptibility Loci on Chromosomes 2q23 and 7p11,” J Invest Dermatol 122:1126-1132 (2004), which are hereby incorporated by reference in their entirety). In contrast, all humans are susceptible to hypertrophic scars. Histologically, keloids demonstrate overgrowth of dense fibrous tissue, extending beyond the borders of the original wound with large thick collagen fibers composed of numerous fibrils closely packed together (Ehrlich et al., “Morphological and Immunochemical Differences Between Keloid and Hypertrophic Scar,” Am J Pathol 145:105-113 (1994); Lee et al., “Histopathological Differential Diagnosis of Keloid and Hypertrophic Scar,” Am J Dermatopathol 26:379-384 (2004); Brissett et al., “Scar Contractures, Hypertrophic Scars, and Keloids,” Facial Plast Surg 17:263-272 (2001); Santucci et al., “Keloids and Hypertrophic Scars of Caucasians Show Distinctive Morphologic and Immunophenotypic Profiles,” Virchows Arch 438:457-463 (2001); Tuan et al., “The Molecular Basis of Keloid and Hypertrophic Scar Formation,” Mol Med Today 4:19-24 (1998), which are hereby incorporated by reference in their entirety).

Murine scars caused by mechanical strain recapitulate all of the classic histopathological features of human hypertrophic scarring. For a comparison of human hypertrophic scarring to murine scars produced by application of mechanical strain according to the present invention, see Table 1, below, and FIGS. 3B-H (Linares et al., “The Histiotypic Organization of the Hypertrophic Scar in Humans,” J Invest Dermatol 59:323-331 (1972); White, C., Textbook of Dermatopathology. New York: McGraw Hill. 349-355 pp. (2004); Ehrlich et al., “Morphological and Immunochemical Differences Between Keloid and Hypertrophic Scar,” Am J Pathol 145:105-113 (1994); Lee et al., “Histopathological Differential Diagnosis of Keloid and Hypertrophic Scar,” Am J Dermatopathol 26:379-384 (2004); Santucci et al., “Keloids and Hypertrophic Scars of Caucasians Show Distinctive Morphologic and Immunophenotypic Profiles,” Virchows Arch 438:457-463 (2001), which are hereby incorporated by reference in their entirety). The similarity between murine scars and human hypertrophic scars are clearly seen in FIGS. 3B-H. The murine scars are grossly and histologically raised, as shown in FIG. 1D and FIG. 3B, respectively. The epidermis overlying the murine hypertrophic scars is flattened. Adnexal structures and hair follicles are absent in the dermis, as shown in FIG. 3C. Hyperplasia occurs in the strained scars, as shown in FIG. 3D. Collagen is arranged in a compact and parallel manner to the skin surface, as shown in FIG. 3E, and the fibroblasts run parallel with the collagen fibers, as shown in FIG. 3F. As early as one week of strain, the mechanically strained wounds demonstrate blood vessels that course perpendicularly towards the epithelium, as shown in FIG. 3G. This is a feature of hypertrophic scars, but not of unstrained wounds or keloids. Collagen whorls/nodules, often seen in chronic human hypertrophic scars (Linares et al., “The Histiotypic Organization of the Hypertrophic Scar in Humans,” J Invest Dermatol 59:323-331 (1972); Ehrlich et al., “Morphological and Immunochemical Differences Between Keloid and Hypertrophic Scar,” Am J Pathol 145:105-113 (1994); Santucci et al., “Keloids and Hypertrophic Scars of Caucasians Show Distinctive Morphologic and Immunophenotypic Profiles,” Virchows Arch 438:457-463 (2001), which are hereby incorporated by reference in their entirety), were also present in the murine model, as shown in FIG. 3H.

TABLE 1

| Scar Characteristics* | Hypertrophic Scars | Keloids |
|---|---|---|
| Loss of rete pegs, adnexae, | Yes | Yes |
| and hair follicles (FIG. | | |
| 3C). | | |
| Increased number of | Yes | No |
| fibroblasts (FIG. 3D). | | |
| Fibrillary collagen is | Yes | No |
| arranged parallel to the skin | | |
| surface (FIG. 3E). | | |
| Increased number of | Yes | No |
| fibroblasts run parallel with | | |
| the fibers (FIG. 3F). | | |
| Blood vessels are arranged | Yes | No |
| perpendicular to the skin | | |
| surface (FIG. 3G). | | |
| Collagenous nodules/whorls | Yes | No |
| in scars (FIG. 3H). | | |
| Large thick collagen | No | Yes |
| fibrils packed closely together. | | |
| Scarring beyond wound margins | No | Yes |
| *(Linares et al., “The Histiotypic Organization of the Hypertrophic Scar in Humans,” J Invest Dermatol 59: 323-331 (1972); White, C., Textbook of Dermatopathology. | | |
| # New York: McGraw Hill. 349-355 pp. (2004); Ehrlich et al., “Morphological and Immunochemical Differences Between Keloid and Hypertrophic Scar,” Am J Pathol 145: 105-113 (1994); | | |
| # Lee et al., “Histopathological Differential Diagnosis of Keloid and Hypertrophic Scar,” Am J Dermatopathol 26: 379-384 (2004); Santucci et al., “Keloids and Hypertrophic Scars of | | |
| # Caucasians Show Distinctive Morphologic and Immunophenotypic Profiles,” Virchows Arch 438: 457-463 (2001); Tuan et al., “The Molecular Basis of Keloid and Hypertrophic Scar | | |
| # Formation,” Mol Med Today 4: 19-24 (1998), which are hereby incorporated by reference in their entirety). | | |

### Example 13

### Mechanical Strain-Disrupts Normal Apoptosis During Proliferative Wound Healing Via the P13-Kinase/Akt

The cellular density per square millimeter of the scars was consistently higher in the hypertrophic scar region than in control scar at all time points, as shown in FIGS. 4D-F. This hyperplasia could theoretically be caused by decreased apoptosis, increased proliferation, or recruitment of stem and/or inflammatory cells.

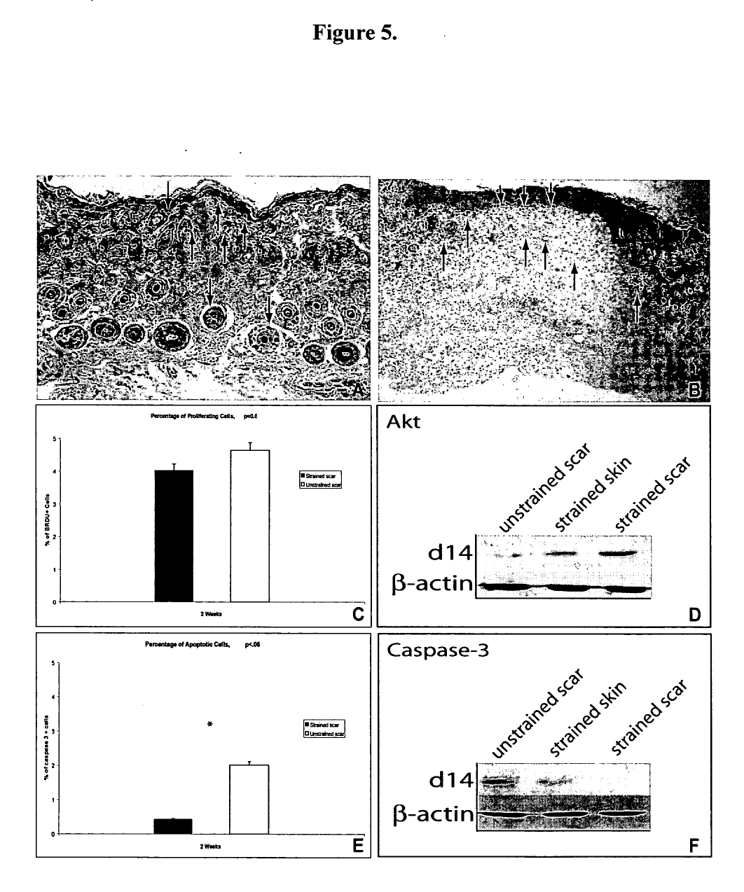

The down-regulation of apoptosis was studied for its possible role in the hypercellularity observed in the strained wounds. Cleaved-caspase 3 immunohistochemistry demonstrated nearly 5-fold down-regulation of apoptosis in the strained scars over the controls (P<0.05). Western blots demonstrated 10-fold and 3-fold less expression in mechanically strained wounds and skin, respectively, than control scars by two weeks (P<0.05), as shown in FIG. 5. Caspases (cysteinyl-directed aspartate-specific proteases) play central roles in apoptosis by initiating the apoptotic cascade (caspase-2, -8, -9, -10), propagating the apoptotic signal (-3, -6, -7) and processing cytokines (-1, -4, -5, -11 to −14). Caspase 3 is a downstream marker of apoptosis, but does not explain how mechanical strain leads to down-regulation of apoptosis. The pro-survival P13-kinase/Akt pathway has been implicated in mechanotransduction. Therefore, its role in hypertrophic scarring was studied. Akt data confirmed the caspase 3 findings. By two weeks, Akt was upregulated 10-fold and 4-fold in the mechanically strained wounds and skin, respectively, versus the control scars (P<0.05), as shown in FIG. 5D. Focal adhesion kinase (“FAK”) localizes to sites of transmembrane integrin receptor clustering and facilitates intracellular signaling events. The P13-kinase/Akt pathway is activated by actin stabilization and FAK upregulation, and the data here demonstrate that mechanical strain leads to activation of this pathway and subsequent hypertrophic scarring.

Proliferation data was not as remarkable as the apoptosis findings and not statistically significant (P>0.05). Normalized BRDU data over four weeks demonstrated only a 1.1-fold difference in overall proliferation between the hypertrophic scar and control scar 870 (217 mean) and 947 (245 mean), shown in FIG. 5C. Furthermore, proliferation was primarily localized to the periphery of the wound margins, epidermis, and hair follicles, as shown in FIGS. 5A-B.

### Example 14

### Mechanical Strain Downregulates Apoptosis in Fibroblasts In Vitro, Induces Other Genes Specific to Matrix Remodeling

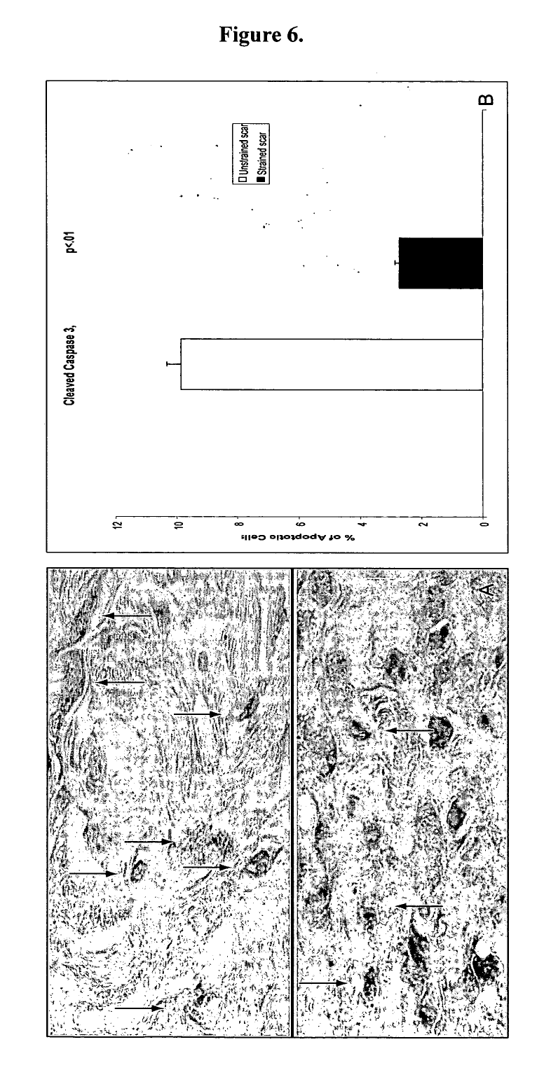

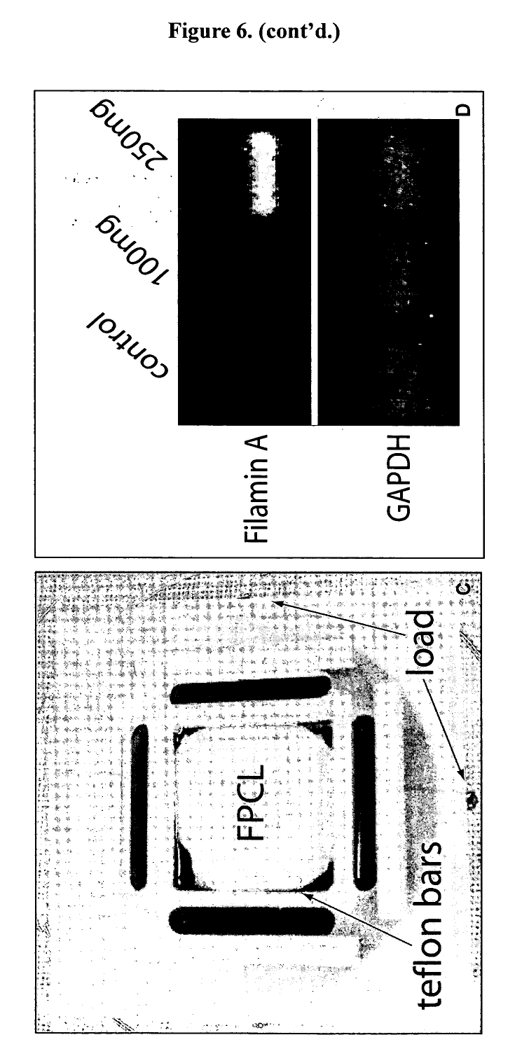

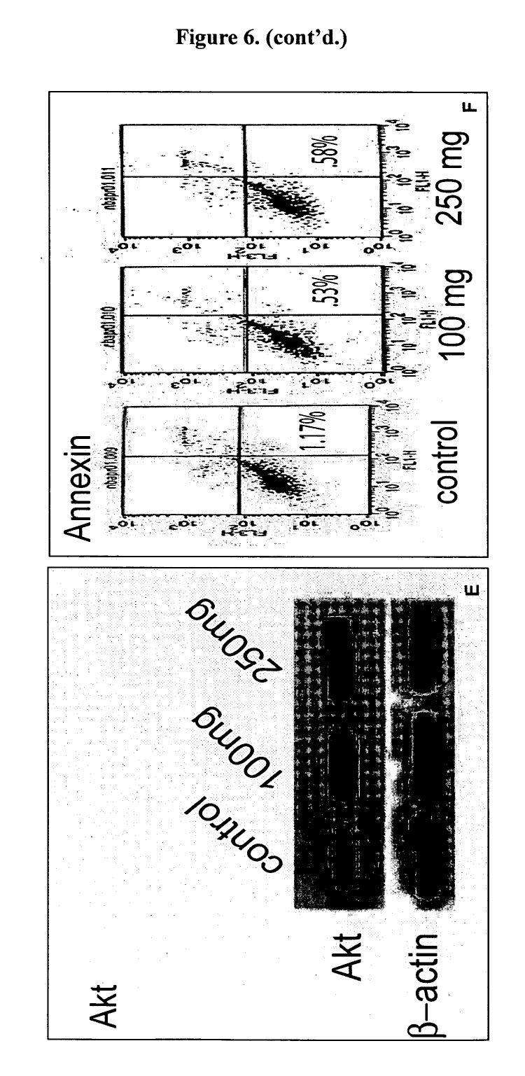

In order to confirm mechanical strain induced down-regulation of apoptosis in fibroblasts and to isolate the effects of mechanical strain outside of the wound healing environment, fibroblast activity was examined in a unique load device. This device, shown in FIG. 6C, applies mechanical strain in a graded fashion to fibroblasts embedded in a 3-D collagen matrix (Knezevic et al., “Isotonic Biaxial Loading of Fibroblast-Populated Collagen Gels: A Versatile, Low-Cost System for the Study of Mechanobiology,” Biomech Model Mechanobiol 1:59-67 (2002), which is hereby incorporated by reference in its entirety). FIG. 6A shows there are fewer fibroblasts in the unstrained scar (top panel), but a greater percentage of cells express caspase-3 antibody signal than in the strained fibroblasts (bottom panel). A 5-fold greater number of caspase 3 positive cells were seen in the unstrained scar than in the strained scar, shown in FIG. 6B. Prior published data show that there is a load-dependent variability in cell survival, cytoskeletal stabilization, and synthesis. Qualitative RT-PCR of Filamin A, an actin-cross-linking protein that stabilizes cell membranes and plays a protective role against force-induced apoptosis (D'Addario et al., “Regulation of Tension-Induced Mechanotranscriptional Signals by the Microtubule Network in Fibroblasts,” J Biol Chem 278:53090-53097 (2003), which is hereby incorporated by reference in its entirety), demonstrated nearly two-fold increase in the relative number of filamin A transcripts, as shown in FIG. 6D. The effects of graded mechanical strain on apoptosis were examined. Akt protein expression was upregulated over two-fold from the control to 250 mg, as shown in FIG. 6E, and annexin V decreased two-fold from the control to 250 mg, as shown in FIG. 6F. Annexin V is a calcium-dependent phospholipid binding protein with high affinity for phosphatidylserine (PS), a membrane component normally localized to the internal face of the cell membrane. Early in the apoptotic pathway, molecules of PS are translocated to the outer surface of the cell membrane where annexin V can readily bind them.

### Example 15

### Altered Apoptotic Pathways Affect Scar Hypertrophy in Knockout Mice

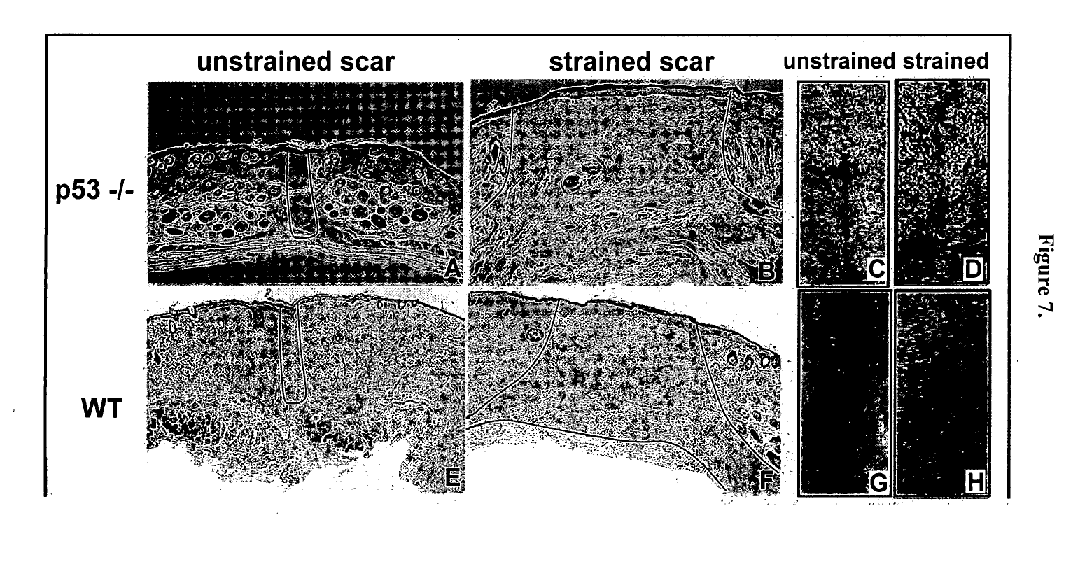

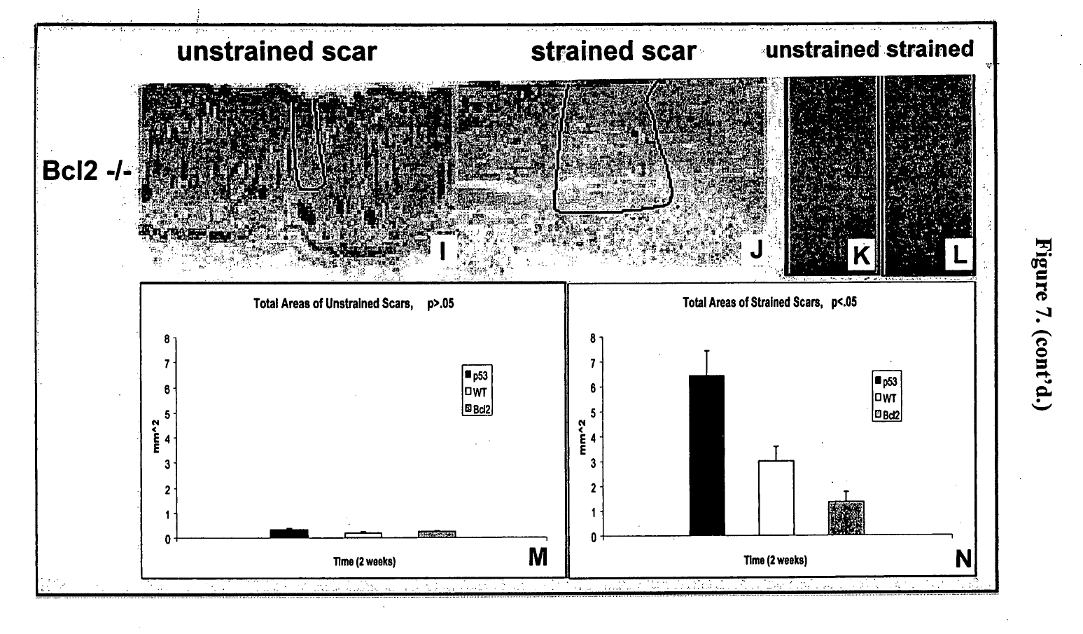

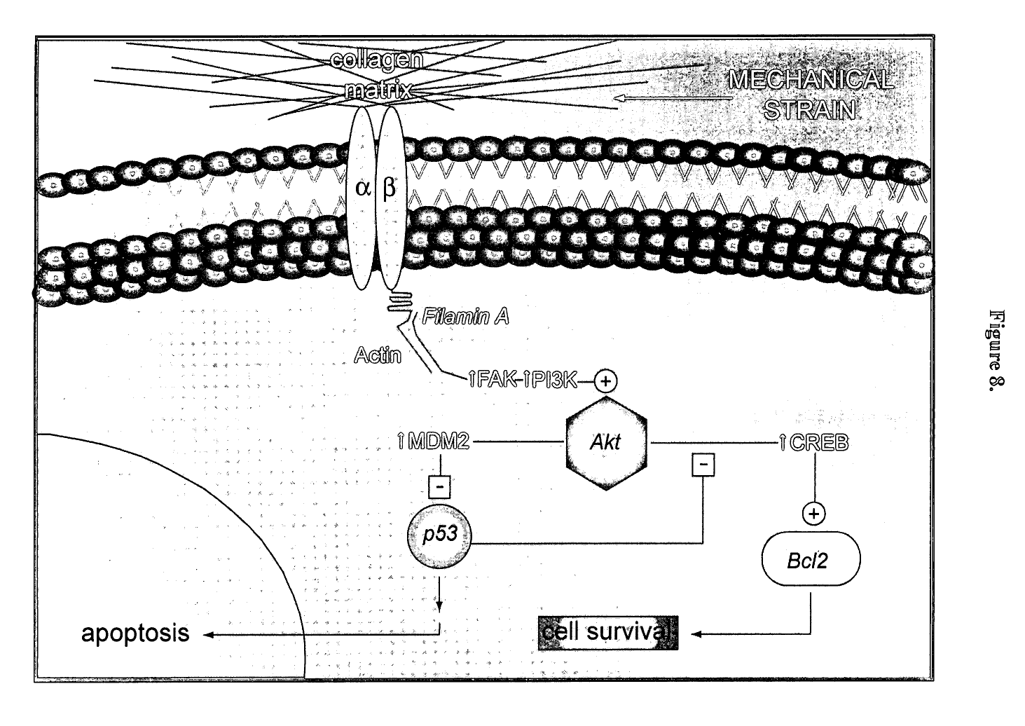

Akt affects other downstream pro- and anti-apoptotic molecules, whose loss may affect the pathophysiology of mechanical strain on healing wounds. Therefore, the role of apoptosis in vivo was further examined in mice lacking specific molecules downstream from Akt. Akt pathway inhibits the pro-apoptotic molecule Bax, upregulates Bcl2 activity and decreases apoptosis (Tsuruta et al., “The Phosphatidylinositol 3-Kinase (PI3K)-Akt Pathway Suppresses Bax Translocation to Mitochondria,” J Biol Chem 277:14040-14047 (2002), which is hereby incorporated by reference in its entirety). Furthermore, Akt has also been shown to decrease apoptosis by directly upregulating cyclic AMP-related binding protein (CREB) which in turn upregulates Bcl2 (Pugazhenthi et al., “Akt/Protein Kinase B Up-Regulates Bcl-2 Expression Through cAMP-Response Element-Binding Protein,” J Biol Chem 275:10761-10766 (2000), which is hereby incorporated by reference in its entirety). A diagram of the P13K/Akt pathway is shown in FIG. 8. The loss of the Bcl2 gene appears to be tantamount to blocking the pro-survival effects of the Akt pathway (Flusberg et al., “Cooperative Control of Akt Phosphorylation, bcl-2 Expression, and Apoptosis by Cytoskeletal Microfilaments and Microtubules in Capillary Endothelial Cells,” Mol Biol Cell 12:3087-3094 (2001), which is hereby incorporated by reference in its entirety). This is demonstrated in the Bcl2 null mice where, despite the pro-survival effects of mechanical strain, hypertrophic scarring was significantly mitigated, shown in FIG. 71 and FIG. 7L. Akt also activates MDM2, which then inhibits p53 (Oren et al., “Regulation of p53: Intricate Loops and Delicate Balances,” Ann N Y Acad Sci 973:374-383 (2002); Gottlieb et al., “Cross-Talk Between Akt, p53 and Mdm2: Possible Implications for the Regulation of Apoptosis,” Oncogene 21:1299-1303 (2002), which are hereby incorporated by reference in their entirety). In the p53 null mouse, the global decrease in apoptosis resulted in larger hypertrophic scars than in the control and Bcl2 null mice. This can be seen by comparing FIGS. 7B and 7D (p53−/− strained tissue) with FIGS. 7F and 7H (wild type strained tissue) and FIGS. 7J and 7L (Bcl2 strained tissue). The strained scars in Bcl2−/− varied from 0.3 to 1.4 mm², and from 4.3 to 7.0 mm² in p53−/− (p<0.05), while the unstrained scars ranged from 0.12 to 0.24 mm² in Bcl2−/−, and from 0.2 to 0.37 mm² in p53−/−(p>0.05). It was concluded from this data that hypertrophic scarring is, in large part, due to decreased apoptosis, and that loss of the Bcl2 pathway results in significant reduction in hypertrophic scarring.

Breaking strengths of strained scars were used as an endpoint marker of wound maturity. The breaking strengths of the strained scars were evaluated at the one week time points in the control and Bcl2−/− mice (pro-apoptotic). There was no difference in wound strength between the two (23.4 N/mm² (control) vs. 22.4 N/mm² (Bcl2−/−, p>0.05)). This suggests that, while there are differences in total scar deposition between the control and Bcl2−/− (see below), scar maturation occurs at the same rate.

Hypertrophic scars, which result in enormous morbidity in truly pathologic conditions such as burn contractures, have no cure. Steroids, irradiation, and pressure therapy are either erratically effective or associated with significant side effects. The lack of effective treatment is perpetuated by the absence of a reliable, reproducible animal model that would enable extensive investigation into the pathophysiology of hypertrophic scarring. Moreover, limited understanding of the pathophysiology has frustrated attempts to treat hypertrophic scars over the past 30 years with resulting recurrence rates exceeding 75% with current treatment options (Deitch et al., “Hypertrophic Burn Scars: Analysis of Variables,” J Trauma 23:895-898 (1983), which is hereby incorporated by reference in its entirety). Described herein is a murine model of hypertrophic scar formation which reproduces all the cardinal features of human disease. Importantly, it is demonstrated herein that hypertrophic scarring results solely from the application of mechanical strain, mirroring clinical association of hypertrophic scarring to mechanical strain that is seen in patients. The initiation of hypertrophic scar formation correlates with a decrease in cellular apoptosis and is accompanied by a dramatic increase in the pro-survival marker Akt. This study has implications for other mechanosensitive disease processes such as cancer (Ingber, D., “Mechanobiology and Diseases of Mechanotransduction,” Ann Med 35:564-577 (2003), which is hereby incorporated by reference in its entirety), glomerulosclerosis (Riser et al., “Cyclic Stretching of Mesangial Cells Up-Regulates Intercellular Adhesion Molecule-1 and Leukocyte Adherence: a Possible New Mechanism for Glomerulosclerosis,” Am J Pathol 158:11-17 (2001), which is hereby incorporated by reference in its entirety), congestive heart failure (Borer et al., “Myocardial Fibrosis in Chronic Aortic Regurgitation: Molecular and Cellular Responses to Volume Overload,” Circulation 105:1837-1842 (2002); Zhang et al., “The Role of the Grb2-p38 MAPK Signaling Pathway in Cardiac Hypertrophy and Fibrosis,” J Clin Invest 111:833-841 (2003), which are hereby incorporated by reference in their entirety), pulmonary hypertension, and atherosclerosis (Gibbons et al., “The Emerging Concept of Vascular Remodeling,” N Engl J Med 330:1431-1438 (1994), which is hereby incorporated by reference in its entirety), where it is believed that perturbation of the surrounding parenchyma and interference with normal mechanotransduction result in fibrosis, and potentiation of tumor angiogenesis and growth (Tomasek et al., “Myofibroblasts and Mechano-Regulation of Connective Tissue Remodelling,” Nat Rev Mol Cell Biol 3:349-363 (2002), which is hereby incorporated by reference in its entirety).

The PI(3)/Akt pro-survival pathway is thought to be upregulated by integrin and actin mediated activation of focal adhesion kinases (Miranti et al., “Sensing the Environment: a Historical Perspective on Integrin Signal Transduction,” Nat Cell Biol 4:E83-90 (2002), which is hereby incorporated by reference in its entirety). Releasing fibroblasts from mechanical constraints down-regulates Akt expression and increases apoptosis (Carlson et al., “Modulation of FAK, Akt, and p53 by Stress Release of the Fibroblast-Populated Collagen Matrix,” J Surg Res 121:151 (2004), which is hereby incorporated by reference in its entirety). Actin polymerization by mechanical strain and filamin A (D'Addario et al., “Regulation of Tension-Induced Mechanotranscriptional Signals by the Microtubule Network in Fibroblasts,” J Biol Chem 278:53090-53097 (2003), which is hereby incorporated by reference in its entirety), results in FAK activation, which in turn activates the PI3 kinase/Akt pathway (Miranti et al., “Sensing the Environment: A Historical Perspective on Integrin Signal Transduction,” Nat Cell Biol 4:E83-90 (2002), which is hereby incorporated by reference in its entirety), as shown in FIG. 8. These data extend the findings by demonstrating that mechanical strain upregulates Akt and Filamin A in a graded fashion and that cyclical mechanical strain in vivo results in hypertrophic scarring. The application of mechanical strain in a cyclical fashion is important, and it has been shown previously that fixed mechanical strain on wounds using splints does not result in hypertrophic scarring (Galiano et al., “Quantitative and Reproducible Murine Model of Excisional Wound Healing,” Wound Repair Regen 12:485-492 (2004), which is hereby incorporated by reference in its entirety). Akt appears to be a central regulator of both the pro-apoptotic p53 and anti-apoptotic Bcl2 pathways (Flusberg et al., “Cooperative Control of Akt Phosphorylation, bcl-2 Expression, and Apoptosis by Cytoskeletal Microfilaments and Microtubules in Capillary Endothelial Cells,” Mol Biol Cell 12:3087-3094 (2001); Oren et al., “Regulation of p53: Intricate Loops and Delicate Balances,” Ann N Y Acad Sci 973:374-383 (2002); Gottlieb et al., “Cross-Talk Between Akt, p53 and Mdm2: Possible Implications for the Regulation of Apoptosis,” Oncogene 21:1299-1303 (2002), which are hereby incorporated by reference in their entirety). A shift in the balance of these pathways would possibly affect the strain induced scar phenotype. The potential balance shifts were studied in vivo using p53 and Bcl2 null mice. The loss of the p53 and Bcl2 pathways resulted in significant augmentation or mitigation, respectively, of strain related survival effects of Akt on hypertrophic scarring.

Mechanical strain is transmitted to the wounds by natural bodily movements, as well as by the inherent elasticity of skin. It has been known that the loss of elastic fibers in humans results in loose skin (cutis laxa), and less fibrosis (Liu et al., “Elastic Fiber Homeostasis Requires Lysyl Oxidase-Like 1 Protein,” Nat Genet 36:178-182 (2004); Kielty et al., “Elastic Fibres,” J Cell Sci 115:2817-2828 (2002); Kielty et al., “Isolation and Ultrastructural Analysis of Microfibrillar Structures From Foetal Bovine Elastic Tissues. Relative Abundance and Supramolecular Architecture of Type VI Collagen Assemblies and Fibrillin,” J Cell Sci 99 (Pt 4):797-807 (1991); Kielty et al., “Attachment of Human Vascular Smooth Muscles Cells to Intact Microfibrillar Assemblies of Collagen VI and Fibrillin,” J Cell Sci 103 (Pt 2):445-451 (1992), which are hereby incorporated by reference in their entirety). Aging, which results in a natural loss of elasticity, also yields less fibrosis. Studies of fetal skin reveal that the fetal extracellular matrix (ECM) is distinct from adult ECM (Adzick et al., “Cells, Matrix, Growth Factors, and the Surgeon. The Biology of Scarless Fetal Wound Repair,” Ann Surg 220:10-18 (1994), which is hereby incorporated by reference in its entirety), with a higher ratio of type III to type I collagen (Merkel et al., “Type I and Type III Collagen Content of Healing Wounds in Fetal and Adult Rats,” Proc Soc Exp Biol Med 187:493-497 (1988); Hallock et al., “Analysis of Collagen Content in the Fetal Wound,” Ann Plast Surg 21:310-315 (1988), which are hereby incorporated by reference in their entirety), and different elastin (Visconti et al., “Codistribution Analysis of Elastin and Related Fibrillar Proteins in Early Vertebrate Development,” Matrix Biol 22:109-121 (2003), which is hereby incorporated by reference in its entirety), proteoglycan, and glycosaminoglycan synthesis profiles (Mast et al., “Hyaluronic Acid is a Major Component of the Matrix of Fetal Rabbit Skin and Wounds: Implications for Healing by Regeneration,” Matrix 11:63-68 (1991), which is hereby incorporated by reference in its entirety). The data has demonstrated that differences in the mechanical properties of skin, such as elasticity, recoil, and elastin content, correlate with the scarring patterns that are seen in human, adult mouse, and murine fetal skin. Early fetal skin has almost no elastic recoil or resting stress. This suggests that scarless healing in the first trimester fetal skin may be influenced by the unique composition of the extracellular matrix, where embryonic cells are free from significant dynamic mechanical forces.

Much research has focused on inflammation as the sole cause for hypertrophic scarring. Inflammatory mediators, such as IL-1 and TNFα (Saulis et al., “Effect of Mederma on Hypertrophic Scarring in the Rabbit Ear Model,” Plast Reconstr Surg 110:177-183; discussion 184-176 (2002); Fitzpatrick, R., “Treatment of Inflamed Hypertrophic Scars Using Intralesional 5-FU,” Dermatol Surg 25:224-232 (1999); Ehrlich, H., “The Physiology of Wound Healing. A Summary of Normal and Abnormal Wound Healing Processes,” Adv Wound Care 11:326-328 (1998), which are hereby incorporated by reference in their entirety), produced during tissue injury could potentially initiate hypertrophic scar formation. Since inflammation is an integral component of the wound healing process it is difficult to determine how inflammation might play a role in hypertrophic scar formation. Some investigators believe that there may simply be an imbalance in the inflammatory milieu leading to increased scar formation. Other theories propose that normal wound healing is altered by bacterial colonization or suture material leading to hypertrophic scar formation (Fitzpatrick, R., “Treatment of Inflamed Hypertrophic Scars Using Intralesional 5-FU,” Dermatol Surg 25:224-232 (1999); Tredget, E., “Management of the Acutely Burned Upper Extremity,” Hand Clin 16:187-203 (2000); Quan et al., “Circulating Fibrocytes: Collagen-Secreting Cells of the Peripheral Blood. Int J Biochem Cell Biol 36:598-606 (2004); Ricketts et al., “Cytokine mRNA Changes During the Treatment of Hypertrophic Scars With Silicone and Nonsilicone Gel Dressings,” Dermatol Surg 22:955-959 (1996); Xue et al., “Altered Interleukin-6 Expression in Fibroblasts From Hypertrophic Burn Scars,” J Burn Care Rehabil 21:142-146 (2000); Kessler-Becker et al., “Expression of Pro-Inflammatory Markers by Human Dermal Fibroblasts in a Three-Dimensional Culture Model is Mediated by an Autocrine Interleukin-1 Loop,” Biochem J 379:351-358 (2004); Polo et al., “The 1997 Moyer Award. Cytokine Production in Patients with Hypertrophic Burn Scars,” J Burn Care Rehabil 18:477-482 (1997); Niessen et al., “The Role of Suture Material in Hypertrophic Scar Formation: Monocryl vs. Vicryl-Rapide,” Ann Plast Surg 39:254-260 (1997), which are hereby incorporated by reference in their entirety). Yet it has not been possible to produce an animal model of hypertrophic scarring based entirely on inflammation, infection, or foreign body contamination. This study demonstrates that wounded skin experiencing negligible mechanical strain does not progress to hypertrophic scarring. On the other hand, the data show that the presence of immortalized macrophages in the Bcl2 null unstrained wound, where there is minimal fibrosis, results in the restoration of the fibrotic response; interestingly, in the Bcl2 null strained wound with the same number of macrophages, restoration of the hypertrophic scar phenotype is seen. This suggests that mechanical strain has an effect that is additive to the effects of macrophages and results in hypertrophic scarring. Unwounded skin was also strained, which resulted in no hypertrophic scarring; again, this highlights the importance of the interaction between mechanical strain and inflammation. It is possible that mechanical strain may prolong the presence of inflammatory cells such as macrophages, or stimulates those cells to overproduce pro-fibrotic growth factors.

This data also demonstrates that the pathophysiology of hypertrophic scarring is highly dependent upon the timing of mechanical strain. Notably, mechanical strain on epithelialized wounds for as brief a period as one week resulted in significant increases in scar cellularity and collagen deposition. In addition, mechanical strain, during the proliferative phase of wound healing, unlike during the inflammatory or remodeling phase, resulted in hypertrophic scarring. These findings have been difficult to elucidate clinically, but suggest the existence of a therapeutic window (before the proliferative phase).

The frustrations of delayed treatment for hypertrophic scars are well appreciated (Mustoe et al., “International Clinical Recommendations on Scar Management,” Plast Reconstr Surg 110:560-571 (2000), which is hereby incorporated by reference in its entirety). Most current therapies, in addition to a nonspecific mode of action, are administered after the hypertrophic scars have matured, when the patient first presents the clinician with the problem. Therapeutic goals would be to develop agents comprising pro-apoptotic molecules such as BH3I-1/BH3I-2, which would block the pro-survival effects of Akt and the related anti-apoptotic activity of Bcl2 family members (Degterev et al., “Identification of Small-Molecule Inhibitors of Interaction Between The BH3 Domain and Bcl-xL,” Nat Cell Biol 3:173-182 (2001), which is hereby incorporated by reference in its entirety), and would be applied to the murine wounds prior to the onset of strain. Clinically, these agents could potentially be applied at the time of wound closure, or after the initial debridement of burn wounds, prior to the onset of the proliferative phase. The concern of diminished breaking strength would have to be addressed, and the time when sutures are released from wounds would need to be re-evaluated; however, the data show that Bcl2−/− and control mice, at one week, demonstrate similar breaking strengths. This suggests that pro-apoptotic therapy may prove to be efficacious in abating hypertrophic scar formation while maintaining adequate wound closure.

The findings here have important implications for fibrotic disorders and tumor growth (Ingber, D., “Mechanobiology and Diseases of Mechanotransduction,” Ann Med 35:564-577 (2003), which is hereby incorporated by reference in its entirety), where disturbed mechanotransduction plays a central role in pathogenesis. It can thus be concluded that mechanical strain potentiates the effects of an inflammatory milieu. Molecular agents that uncouple the transduction of mechanical strain at the level of integrins or intracellularly could prove to be a useful therapeutic modality. A logical step forward in this work would be to investigate the role of bone marrow derived inflammatory or stem cells in hypertrophic scarring. Identifying precisely when such cells are mobilized to the strained scar would clarify the parameters of the therapeutic window.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of producing a non-human animal model of hypertrophic scarring, said method comprising:
producing an incision in a non-human animal and
applying mechanical strain over the incision under conditions effective to produce hypertrophic scarring, thereby producing a non-human animal model of hypertrophic scarring.

2. The method according to claim 1, wherein said mechanical strain is applied by attaching a device to the animal, wherein the device is capable of providing mechanical strain over the incision in one or more directions relative to the incision's direction.

3. The method according to claim 1, wherein the hypertrophic scarring produced comprises the characteristics of human hypertrophic scarring.

4. The method according to claim 3 further comprising:
alternating said applying of the mechanical strain over the incision with periods of relaxation of the mechanical strain over the incision.

5. The method according to claim 2, wherein the mechanical strain is applied in one direction relative to the direction of the incision.

6. The method according to claim 5, wherein the mechanical strain is applied parallel to the direction of the incision.

7. The method according to claim 5, wherein the mechanical strain is applied perpendicular to the direction of the incision.

8. The method according to claim 2, wherein the mechanical strain is applied in more than one direction relative to the direction of the incision.

9. The method according to claim 1, wherein the animal is a rodent.

10. The method according to claim 9, wherein the rodent is a mouse.

11. The non-human animal model produced by the method of claim 1.

12. A method of determining the efficacy of an agent for prevention or treatment of a disease condition, said method comprising:
providing a non-human animal having an incision over which mechanical strain is applied under conditions effective to produce hypertrophic scarring;
administering an agent to the incision; and
determining whether the agent is efficacious for prevention or treatment of a disease condition.

13. The method according to claim 12, wherein said mechanical strain is applied by attaching a device to the animal, wherein the device provides mechanical strain over the incision in one or more directions relative to the incision's direction.

14. The method according to claim 12 further comprising:
alternating said applying of mechanical strain over the incision with periods of relaxation of the mechanical strain over the incision.

15. The method according to claim 13, wherein the mechanical strain is applied in one direction relative to the direction of the incision.

16. The method according to claim 15, wherein the mechanical strain is applied parallel to the direction of the incision.

17. The method according to claim 15, wherein the mechanical strain is applied perpendicular to the direction of the incision.

18. The method according to claim 13, wherein the mechanical strain is applied in more than one direction relative to the direction of the incision.

19. The method according to claim 12, wherein the animal is a rodent.

20. The method according to claim 19, wherein the rodent is a mouse.

21. The method according to claim 12, wherein the agent is efficacious where there is a decrease in hypertrophic scarring in the non-human animal model receiving the agent compared to a hypertrophic scarring animal model that has not received the agent.

22. The method according to claim 12, wherein said administering is carried out dermally.

23. The method according to claim 22, wherein said administering is carried out prior to the incision entering a proliferative phase of wound healing.

24. The method according to claim 12, wherein the agent is a pro-apoptotic agent.

25. The method according to claim 24, wherein the pro-apoptotic agent is BH3I-1/BH3I-2.

26. The method according to claim 12, wherein the agent blocks the activity of anti-apoptotic molecules.

27. The method according to claim 12, wherein the disease condition is hypertrophic scarring, a fibrotic disorder, cancer tumors, glomerulosclerosis, congestive heart failure, cardiac hypertrophy, Dupytren's contracture, pulmonary hypertension, or atherosclerosis.

28. The method according to claim 27, wherein the disease condition is hypertrophic scarring.

29. A non-human animal model of hypertrophic scarring comprising a non-human animal having an incision over which mechanical strain has been applied under conditions effective to produce hypertrophic scarring.

30. The non-human animal model of hypertrophic scarring according to claim 29, wherein the hypertrophic scarring comprises the characteristics of human hypertrophic scarring.

31. The non-human animal model according to claim 29, wherein the animal is a rodent.

32. The non-human animal model according to claim 31, wherein the rodent is a mouse.
